(19) United States
(12) Patent Application Publication
(10) Pub. No.: US 2019/0016818 A1
(43) Pub. Date: Jan. 17, 2019

(54) ANTIBODIES AGAINST GLYPICAN-3 AND THEIR USES IN CANCER DIAGNOSIS AND TREATMENT

(71) Applicants: LA JOLLA BIOLOGICS, INC., San Diego, CA (US); TANVEX BIOLOGICS CORP., New Tapei City (TW)

(72) Inventors: Yun YEN, Arcadia, CA (US); Yu-Ching LEE, Taipei City (TW); Yi-Yuan YANG, Taipei City (TW)

(21) Appl. No.: 15/577,312

(22) PCT Filed: May 27, 2016
(86) PCT No.: PCT/US16/34633

§ 371 Date: Nov. 27, 2017

Related U.S. Application Data
(60) Provisional application No. 62/166,760, filed on May 27, 2015.

(51) Int. Cl. C07K 16/30; C07K 16/00; C07K 16/02; A61P 35/00; G01N 33/574

(52) U.S. Cl. CPC C07K 16/303; C07K 16/005; G01N 2400/40; A61P 35/00; G01N 33/57438; C07K 16/02

(57) ABSTRACT

The present invention relates to anti-GPC3 antibodies and their applications. The invention investigates the potential inhibitory effect of anti-GPC3 antibodies on tumor growth, proliferation, migration and their applications for diagnostic and therapeutic purposes.

### RELATED APPLICATIONS

This application is a § 371 National Phase Application of International Application No. PCT/US2016/034633, filed on May 27, 2016, which claims the benefit of U.S. Provisional Patent Application No. 61/166,760, filed on May 27, 2015, which is incorporated herein by reference.

### FIELD OF THE INVENTION

The present invention relates to antibodies for cancer diagnosis and treatment. Particularly, the present invention relates to antibodies against glypican-3 (GPC3) and uses in treatment of cancer.

### BACKGROUND OF THE INVENTION

Glypican-3 (GPC3) is a cell surface protein that is highly expressed in HCC and some other human cancers including melanoma. The GPC3 gene encodes a 70-kDa precursor core protein with 580 amino acids, which can be cleaved by furin to generate a 40-kDa amino (N) terminal protein and a 30-kDa membrane-bound carboxyl (C) terminal protein, which has two heparan sulphate (HS) glycan chains. Six glypicans (GPC1-6) have been identified in mammals. All glypicans share a characteristic structure. These common features suggest that glypicans may share a similar three-dimensional (3D) structure.

An interaction between GPC3 and FGF-2 has also been found in HCC cells (Midorikawa, Y. et al., (2003). International journal of cancer Journal international du cancer 103, 455-465). It has been hypothesized that a mutated GPC3 lacking the GPI anchoring domain can block Wnt signaling and inhibit the growth of Wnt-dependent tumors. However, one cannot rule out the possibility that inhibition of HCC growth may be due to the activity of other factors such as heparin binding growth factors modulated by the HS chains. Hepatocellular carcinoma (HCC) and Cholangiocarcinoma (CCA) are the two major forms of primary liver cancer. A growing body of evidence support that GPC3 is a new tumor marker for HCC. GPC3 is highly expressed in the HCC cell lines, HepG2, Hep3B, HT17, HuH6, HuH7 and PLC/PRF/(Song, H. H. et al., (2005). The Journal of biological chemistry 280, 2116-2125). In addition, GPC3 is highly expressed in HCC (Hsu, H. C., Cheng, W, and Lai, P. L. (1997). Cancer research 57, 5179-5184) but not in CCA or normal liver tissue. GPC3 is also expressed to a lesser degree in melanoma (Nakatsura, T et al., (2004a). Clinical cancer research: an official journal of the American Association for Cancer Research 10, 6612-6621), ovarian clear-cell carcinomas (Stadlmann, S., Gueth et al., (2007). Clinical cancer research: an official journal of the International Society of Gynecological Pathologists 26, 341-344), yolk sac tumors (Zynger, D. L et al., (2006). The American journal of surgical pathology 30, 1570-1575), neuroblastoma, hepatoblastoma, Wilms' tumor cells and other tumors (Baumhoer, D., Tornillo et al., (2008). American journal of clinical pathology 129, 899-906; Saikali, Z., and Sinnett, D. (2000). International journal of cancer Journal international du cancer 89, 418-422). On the other hand, GPC3 is silenced in breast cancer, mesothelioma, epithelial ovarian cancer and lung adenocarcinoma. GPC3 protein expression is found in more than 70% of HCC tumors but not in normal liver tissue when using a rabbit polyclonal antibody raised against human GPC3 (residues 303-464) (Nakatsura, T, et al. (2003). Biochemical and biophysical research communications 306, 16-25). Due to the finding that GPC3-positive HCC patients have a significantly lower 5-year survival rate than GPC3-negative HCC patients, GPC3 expression is correlated with poor prognosis in HCC (Shirakawa, H. et al. (2009). Cancer science 100, 1403-1407). Since it shows high expression in HCC, GPC3 has a potential as a promising target for tumor-specific therapy. Also, because small amounts of GPC3 can be detected in the blood of some patients with GPC3-positive cancers (Capurro, M et al., (2003). Gastroenterology 125, 89-97; Hippo, Y. et al., (2004). Cancer research 64, 2418-2423), measurement of GPC3 in the blood may be a useful diagnostic to follow the course of these patients.

Given the high expression of GPC3 in HCC, melanoma and clear cell carcinomas of the ovary, the usefulness of GPC3 as a potential candidate for both antibody- and cell-based immunotherapies has been evaluated. In 2003, a mAb against a GPC3 peptide consisting of 17 residues (355-371) was reported to study the interaction of GPC3 and FGF-2 (Midorikawa, Y. et al., (2003). International journal of cancer Journal international du cancer 103, 455-465). Subsequently, a mAb (IgG1, κ) specific for the last 70 amino acids of the C terminus of the GPC3 protein (Capurro, M et al., (2003). Gastroenterology 125, 89-97) and two mAbs specific for the residues 25 to 358 of GPC3 were generated and used to detect serum GPC3 in HCC patients (Hippo, Y., Watanabe et al., (2004). Cancer research 64, 2418-2423). Although both laboratories used the mAbs with two different terminal groups, they found a similar proportion of GPC3-positive sera in HCC patients. In addition, Yamauchi et al. used the GPC3 protein lacking the GPI anchor as an immunogen to obtain two mAbs for the N terminus of GPC3 and for the C terminus, respectively. These mAbs were used for immune-histochemical analysis of cancer (Yamauchi, N. et al., (2005). Modern pathology: an official journal of the United States and Canadian Academy of Pathology, Inc 18, 1591-1598).

The first therapeutic mAb recognizing residues 524 to 563 of GPC3 has recently been described (Ishiguro, T. et al., (2008). Cancer research 68, 9832-9838; Nakano, K. et al., (2009). Biochemical and biophysical research communications 378, 279-284). The mAb, designated GC33, induced antibody-dependent cellular cytotoxicity (ADCC) and exhibited tumor growth inhibition of subcutaneous transplanted HepG2 and HuH-7 ectopic xenografts in mice. GC33 also reduced the blood α-fetoprotein levels of mice intrahepatically transplanted with HepG2 cells in an orthotopic model. Humanized GC33 (hGC33) is as effective as GC33 against the HepG2 xenograft (Nakano, K., Ishiguro et al., (2010). Anti-cancer drugs 21, 907-916). The ADCC anti-tumor activity of GC33 is mainly due to natural killer cells (Ishiguro, T., Sugimoto et al., (2008). Cancer research 68, 9832-9838). On the other hand, Takai et al. investigated the relationship between membrane expression of GPC3 and recruitment of tumor-associated macrophage (TAM) (Takai, H. et al., (2009a). Cancer biology & therapy 8, 2329-2338; Takai, H. et al., (2009b). Liver international: official journal of the International Association for the Study of the Liver 29, 1056-1064). They observed the involvements of infiltrated TAM in anti-GPC3 immunotherapy model using GC33, showing macrophages may play an important role in the anti-tumor activity of GC33 by non-ADCC mechanisms such as modulation of GPC3 functions (Takai, H et al., (2009c). Cancer biology & therapy 8, 930-938). In addition, GC33 does not directly inhibit the proliferation of GPC3-positive tumor cells. To fully evaluate GPC3-targeted antibody therapy, the mAbs that target different functional domains (including the HS chain) of GPC3 would be useful. It would be interesting to investigate the anti-tumor activity of the anti-GPC3 mAbs that are able to directly inhibit cancer cell proliferation and/or survival by blocking Wnt and/or other signaling pathways.

### SUMMARY OF THE INVENTION

The present invention is at least based on the finding that the functional domain or antigenic epitopes present in GPC3 protein can serve as a potential target for diagnostic and/or therapeutic application. Accordingly, aspects of the present invention characterize the anti-GPC3 antibodies and demonstrates the potential inhibitory effect of anti-GPC3 antibodies on tumor growth, proliferation, migration and their applications for diagnostic and therapeutic purposes. Particularly, hepatocellular carcinoma (HCC) remains a common malignant cancer worldwide. There is an urgent need to identify new molecular targets for the development of novel therapeutic approaches. The present invention surprisingly found that GPC3 is a promising candidate for liver cancer therapy given that it shows high expression in HCC. Herein, it is shown that membrane-bound PGC3 molecule is a therapeutic target for immunotherapy and soluble GPC3 may be a useful serum biomarker for HCC.

In one aspect, the present invention provides an isolated anti-GPC3 antibody and/or an antigen-binding portion thereof, comprising at least one of a heavy chain complementarity determining region 1 (H-CDR1) comprising the amino acid residue of SEQ ID NO: 1, 2, 3 or 4, or a variant having amino acid sequence with at least 80%, 85%, 90%, 91%, 92%, 93%, 94%, 95%, 96%, 97%, 98%, 99% identity to any of SEQ ID NOs: 1 to 4; a heavy chain CDR2 (H-CDR2) comprising the amino acid residue of SEQ ID NO: 5, 6, 7, 8 or 9, or a variant having amino acid sequence with at least 80%, 85%, 90%, 91%, 92%, 93%, 94%, 95%, 96%, 97%, 98%, 99% identity to any of SEQ ID NOs: 5 to 9; and a heavy chain CDR3 (H-CDR3) comprising the amino acid residue of SEQ ID NO: 10, 11, 12, 13 or 14, or a variant having amino acid sequence with at least 80%, 85%, 90%, 91%, 92%, 93%, 94%, 95%, 96%, 97%, 98%, 99% identity to any of SEQ ID NOs: 10 to 14; and at least one of a light chain CDR1 (L-CDR1) comprising the amino acid residue of SEQ ID NO: 15, 16 or 17, or a variant having amino acid sequence with at least 80%, 85%, 90%, 91%, 92%, 93%, 94%, 95%, 96%, 97%, 98%, 99% identity to any of SEQ ID NOs: 15 to 17; a light chain CDR2 (L-CDR2) comprising the amino acid residue of SEQ ID NO: 18, 19, 20, 21 or 22, or a variant having amino acid sequence with at least 80%, 85%, 90%, 91%, 92%, 93%, 94%, 95%, 96%, 97%, 98%, 99% identity to any of SEQ ID NOs: 18 to 22; and a light chain CDR3 (L-CDR3) comprising the amino acid residue SEQ ID NO: 23, 24, 25, 26 or 27, or a variant having amino acid sequence with at least 80%, 85%, 90%, 91%, 92%, 93%, 94%, 95%, 96%, 97%, 98%, 99% identity to any of SEQ ID NOs: 23 to 27; such that said isolated antibody or antigen-binding portion thereof binds to GPC3.

In some embodiments, the invention provides a heavy chain comprising an amino acid sequence having a sequence selected from the group consisting of the sequences as set forth in SEQ ID NOs: 28 to 35.

In some embodiments, the invention provides a light chain comprising an amino acid sequence having the sequence selected from the group consisting of those as set forth in SEQ ID NOs: 36 to 43.

In another aspect, the present disclosure also provides an antibody and/or fragment thereof that binds to GPC3, wherein at least one of the heavy chain CDRs and/or at least one of the light chain CDRs comprises at least one amino acid modification.

In one embodiment, the antibody is humanized scFv antibody. In a further embodiment, the humanized scFv antibody comprises a heavy chain having an amino acid sequence as set forth in SEQ ID NO: 34 and a light chain having an amino acid sequence as set forth in SEQ ID NO: 42 (G5S1 humanized scFv antibody). In another further embodiment, the invention comprises a humanized scFv antibody, comprising a heavy chain having an amino acid sequence as set forth in SEQ ID NO: 35 and a light chain having an amino acid sequence as set forth in SEQ ID NO: 43 (GES1 humanized scFv antibody).

In another aspect, the invention provides a pharmaceutical composition comprising the anti-GPC3 antibody of the invention and a pharmaceutically acceptable carrier or excipient.

In another aspect, the invention provides a method for treating a cancer in a subject in need thereof comprising administering an effective amount of a pharmaceutically acceptable composition comprising the anti-GPC3 antibody of the invention to the subject.

In a further aspect, the invention provides a method for diagnosis of a cancer, comprising detecting the binding of the antibody of the invention to a GPC3 protein in a sample.

In one embodiment, the invention provides a method for diagnosing a cirrhotic liver or liver cancer in a subject, comprising detecting a binding of the antibody of the invention to GPC3 in a biological sample, wherein the binding indicates that there is likelihood of the subject developing a cirrhotic liver and liver cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows analysis results of GPC3_ECD protein.

FIG. 2 shows the binding activity of anti-GPC3 antibodies using ELISA.

FIG. 3 shows analysis of purified scFv antibodies on SDS-PAGE.

FIG. 4 A to C show the heavy chains (A) and light chains (B) of the selected scFv sequences of GPC3 gene of chicken (555 S1, S8 and GPC3 S1, S2, S6, S8) and heavy chains and light chains of humanized scFv sequence of the invention (G5S1 humanized scFv sequence and GES1 humanized scFv sequence).

FIG. 5 A to D show electrophoresis analysis of cell lysates of four hepatoma cell lines (Lanes 1-4) and four sarcomatoid hepatoma cell lines (Lanes 5-8). FIG. 5A shows commercial GPC3 extracellular domain protein under reducing condition (Lane C) and the upper arrow shows C-terminus fragment and the lower arrow shows N-terminus fragment; FIG. 5B shows two fragments identified by anti-GPC3 poly IgY; FIG. 5C shows fragment identified by G5S1 scFv; and FIG. 5D shows fragment identified by GES1 scFv.

FIG. 6 shows the binding analysis of specific anti-GPC3 scFv antibodies on ELISA.

FIGS. 7 A and B show proliferative inhibition of specific anti-GPC3 scFv antibodies on hepatoma cells. FIG. 7A shows proliferative inhibition of specific anti-GPC3 scFv antibodies at different days. FIG. 7B shows proliferative inhibition of specific anti-GPC3 scFv antibodies at different concentrations.

FIG. 8 shows the binding analysis of specific anti-GPC3 scFv antibodies using flow cytometry.

FIG. 9A shows the binding analysis of specific anti-GPC3 scFv antibodies using immunofluoresence staining; Hep 3B cells. FIG. 9B shows the binding analysis of specific anti-GPC3 scFv antibodies using immunofluoresence staining; Hep G2 cells.

FIG. 10 shows the binding analysis of specific anti-GPC3 scFv antibodies using immunoprecipitation analysis.

FIG. 11 shows the inhibition of specific anti-GPC3 scFv antibodies on colony formation assay.

FIGS. 12 A, B and C show the results of cell cycle analysis. The cells were arrested in G1 phase when treated with 0.5 μM of G5S1 and GES1 scFv antibodies (A and B). The cell population in subG1 stage was significantly increased to 28.8% and 16.6% in GES1 and G5S1 treated HepG2 cells, respectively, which may result from the induction of cell apoptosis (C).

FIG. 13 shows the inhibition of specific anti-GPC3 scFv antibodies on cell migration.

FIG. 14A shows the antitumor effects of G5S1 and GES1 against human Hep3B xenograft model. FIG. 14B shows the antitumor effects of G5S1 and GES1 against the body weight of the mice.

FIG. 15 shows immunohistochemical analysis on tumorous tissues in xenographic mice.

FIGS. 16 A and B show that 1 mg/Kg and 5 mg/Kg of GES1 IgG can inhibit tumor growth to 32.4% and 51.2%, respectively, whereas sorafenib only has 48.8% inhibition (A). There is no significant change in body weight of the mice (B).

FIG. 17 A to B show that 10 mg/Kg of GES1 IgG can significantly inhibit tumor growth (p<0.01) (A); the expression levels of p-AKT and p-Erk after antibody treatment decreased (B, B2-2, B2-3 and B2-5); the expression level of Ki-67 protein in tumor tissues treated by GES1 IgG significantly decreased in comparison with those treated by the commercial antibody (C and D).

### DETAILED DESCRIPTION OF THE INVENTION

In the description that follows, a number of terms are used and the following definitions are provided to facilitate understanding of the claimed subject matter. Terms that are not expressly defined herein are used in accordance with their plain and ordinary meaning.

### Definitions

Unless otherwise specified, a or an means “one or more.”

As used herein, the term “epitope” refers to the site on the antigen to which an antibody binds.

As used herein, the term “antibody” refers to single chain, two-chain, and multi-chain proteins and polypeptides belonging to the classes of polyclonal, monoclonal, chimeric, and humanized antibodies; it also includes synthetic and genetically engineered variants of these antibodies. “Antibody fragment” includes Fab, Fab′, F(ab′)₂, and Fv fragments, as well as any portion of an antibody having specificity toward a desired target epitope or epitopes.

As used herein, the term “polyclonal antibody” refers to an antibody which is produced among or in the presence of one or more other, non-identical antibodies. In general, polyclonal antibodies are produced from a B-lymphocyte in the presence of several other B-lymphocytes producing non-identical antibodies. Usually, polyclonal antibodies are obtained directly from an immunized animal.

As used herein, the term “monoclonal antibody” refers to an antibody obtained from a population of substantially homogeneous antibodies. In other words, a monoclonal antibody consists of a homogeneous antibody arising from the growth of a single cell clone (for example a hybridoma, a eukaryotic host cell transfected with a DNA molecule coding for the homogeneous antibody or a prokaryotic host cell transfected with a DNA molecule coding for the homogeneous antibody). These antibodies are directed against a single epitope and are therefore highly specific.

As used herein, “variable domain” refers to the domains that mediate antigen-binding and defines specificity of a particular antibody for a particular antigen. The antigen-binding site consists of two variable domains that define specificity: one located in the heavy chain (VH) and the other located in the light chain (VL). In some cases, specificity may exclusively reside in only one of the two domains as in single-domain antibodies from heavy-chain antibodies found in camelids. The variable domains of native heavy and light chains comprise four FRs, largely adopting a beat-sheet configuration, connected by three hypervariable regions, which form loops. The hypervariable regions in each chain are held together in close proximity by FRs, and with the hypervariable regions from the other chain, contribute to the formation of the antigen binding site of antibodies (see Kabat E A et al., supra). The “hypervariable region” refers to the amino acid residues of an antibody which are responsible for antigen binding. The hypervariable region generally comprises amino acid residues from a “complementary determining region” or “CDR,” the latter being of highest sequence variability and/or involved in antigen recognition. For all variable domains, numbering is according to Kabat (Kabat E A et al., supra).

A number of CDR definitions in use are encompassed herein. The Kabat definition is based on sequence variability (Kabat E A et al., supra). Chothia refers instead to the location of the structural loops (Chothia C & Lesk A M (1987) J. Mol. Biol. 196: 901-917). The AbM definition is used by Oxford Molecular's AbM antibody modelling software (Martin A C R et al., (1989) Proc. Natl. Acad. Sci. USA, 86: 9268-72Oxford University Press, Oxford, 141-172). The contact definition has been recently introduced (MacCallum R M et al., (1996) J. Mol. Biol. 262: 732-745) and is based on an analysis of the available complex structures available in the Protein Databank. The definition of the CDR by the international ImMunoGeneTics information System® (IMGT®). (http://www.imgt.org) is based on the IMGT numbering for all immunoglobulin and T cell receptor V-Regions of all species (IMGT®, the international ImMunoGeneTics information system; Lefranc M P et al., (2005) Dev. Comp. Immunol. 29(3): 185-203; Kaas Q et al., (2007) Briefings in Functional Genomics & Proteomics, 6(4): 253-64.

As used herein, all Complementarity Determining Regions (CDRs) discussed in the present invention are defined preferably according to IMGT®. The variable domain residues for these CDRs are numbered according to IMGT® (Lefranc M P., (1999) The Immunologist. 7: 132-136; Lefranc M P et al., (2003) Dev. Comp. Immunol. 27(1): 55-77)).

As used herein, the term “humanized antibody” refers to a recombinant protein in which the CDRs from an antibody from one species; e.g., a murine or a chicken antibody, are transferred from the heavy and light variable chains of the antibody from the species into human heavy and light variable domains (framework regions). The constant domains of the antibody molecule are derived from those of a human antibody. In some cases, specific residues of the framework region of the humanized antibody, particularly those that are touching or close to the CDR sequences, may be modified, for example replaced with the corresponding residues from the original murine, rodent, subhuman primate, or other antibody. The humanized antibody may be achieved by various methods including (i) grafting only the non-human CDRs onto human framework and constant regions with or without retention of critical framework residues, or (ii) transplanting the entire non-human variable domains, but “cloaking” them with a human-like section by replacement of surface residues. Such methods as are useful in practicing the present invention include that disclosed in Padlan, Mol. Immunol., 31(3): 169-217 (1994).

As used herein, the term “chimeric antibody” refers to a recombinant protein that contains the variable domains of both the heavy and light antibody chains, including the complementarity determining regions (CDRs) of an antibody derived from one species, preferably a rodent antibody or a chicken antibody, more preferably a murine antibody, while the constant domains of the antibody molecule are derived from those of a human antibody.

As used herein, the term “Fv” is a minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. The six HVRs confer antigen-binding specificity to the antibody. However, even a single variable domain (or half of an Fv comprising only three HVRs specific for an antigen) has the ability to recognize and bind an antigen, although at a lower affinity than the entire binding site.

As used herein, the term “diagnostic” or “diagnosed” means identifying the presence or nature of a pathologic condition.

As used herein, the terms “treatment,” “treating,” and the like, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “therapeutically effective amount” or “efficacious amount” refers to the amount of a subject anti-GPC-3 antibody that, when administered to a mammal or other subject for treating a disease, is sufficient to effect such treatment for the disease.

As used herein, the term “biological sample” encompasses a variety of sample types obtained from an individual, subject or patient and can be used in a diagnostic or monitoring assay. The definition encompasses blood and other liquid samples of biological origin, solid tissue samples such as a biopsy specimen or tissue cultures or cells derived therefrom and the progeny thereof.

### Anti-GPC-3 Antibodies

The present invention relates to antibodies against glypican-3 (GPC3) and fragments thereof that bind to GPC3. The term “antibody or fragment thereof that binds to GPC3” as used herein includes antibodies or a fragment thereof that binds to GPC3. An anti-GPC3 antibody may increase the susceptibility of HCC to chemotherapeutic agents (Ishiguro T et al., (2010). Proceedings of the 101st Annual Meeting of the AACR). The combination regimen may be clinically useful as an anti-liver cancer therapy.

In one aspect, the present invention provides an isolated anti-GPC3 antibody or an antigen-binding portion thereof, comprising at least one of a heavy chain complementarity determining region 1 (H-CDR1) comprising the amino acid residue of SEQ ID NO: 1, 2, 3 or 4, or a variant having amino acid sequence with at least 80% identity to any of SEQ ID NOs: 1 to 4; a heavy chain CDR2 (H-CDR2) comprising the amino acid residue of SEQ ID NO: 5, 6, 7, 8 or 9, or a variant having amino acid sequence with at least 80% identity to any of SEQ ID NOs: 5 to 9; and a heavy chain CDR3 (H-CDR3) comprising the amino acid residue of SEQ ID NO: 10, 11, 12, 13 or 14, or a variant having amino acid sequence with at least 80% identity to any of SEQ ID NOs: 10 to 14; and at least one of a light chain CDR1 (L-CDR1) comprising the amino acid residue of SEQ ID NO: 15, 16 or 17, or a variant having amino acid sequence with at least 80% identity to any of SEQ ID NOs: 15 to 17; a light chain CDR2 (L-CDR2) comprising the amino acid residue of SEQ ID NO: 18, 19, 20, 21 or 22, or a variant having amino acid sequence with at least 80% identity to any of SEQ ID NOs: 18 to 22; and a light chain CDR3 (L-CDR3) comprising the amino acid residue SEQ ID NO: 23, 24, 25, 26 or 27, or a variant having amino acid sequence with at least 80% identity to any of SEQ ID NOs: 23 to 27; such that said isolated antibody or antigen-binding portion thereof binds to GPC3. Preferably, the sequence identity as mentioned above is at least 81%, 82%, 83%, 84%, 85%, 86%, 87%, 88%, 89%, 90%, 91%, 91%, 92%, 93%, 94%, 95%, 96%, 97%, 98% or 99%.

In one embodiment, the invention provides an isolated anti-GPC3 antibody or an antigen-binding portion thereof, comprising a heavy chain complementarity determining region 1 (H-CDR1) comprising the amino acid residue of SEQ ID NO: 1, a heavy chain CDR2 (H-CDR2) comprising the amino acid residue of SEQ ID NO: 5 and a heavy chain CDR3 (H-CDR3) comprising the amino acid residue of SEQ ID NO: 10; and a light chain CDR1 (L-CDR1) comprising the amino acid residue of SEQ ID NO: 15, a light chain CDR2 (L-CDR2) comprising the amino acid residue of SEQ ID NO: 18 and a light chain CDR3 (L-CDR3) comprising the amino acid residue of SEQ ID NO: 23.

In one embodiment, the invention provides an isolated anti-GPC3 antibody or an antigen-binding portion thereof, comprising a heavy chain complementarity determining region 1 (H-CDR1) comprising the amino acid residue of SEQ ID NO: 2, a heavy chain CDR2 (H-CDR2) comprising the amino acid residue of SEQ ID NO: 6 and a heavy chain CDR3 (H-CDR3) comprising the amino acid residue of SEQ ID NO: 11; and a light chain CDR1 (L-CDR1) comprising the amino acid residue of SEQ ID NO: 15, a light chain CDR2 (L-CDR2) comprising the amino acid residue of SEQ ID NO: 19 and a light chain CDR3 (L-CDR3) comprising the amino acid residue of SEQ ID NO: 24.

In one embodiment, the invention provides an isolated anti-GPC3 antibody or an antigen-binding portion thereof, comprising a heavy chain complementarity determining region 1 (H-CDR1) comprising the amino acid residue of SEQ ID NO: 3, a heavy chain CDR2 (H-CDR2) comprising the amino acid residue of SEQ ID NO: 7 and a heavy chain CDR3 (H-CDR3) comprising the amino acid residue of SEQ ID NO: 12; and a light chain CDR1 (L-CDR1) comprising the amino acid residue of SEQ ID NO: 16, a light chain CDR2 (L-CDR2) comprising the amino acid residue of SEQ ID NO: 20 and a light chain CDR3 (L-CDR3) comprising the amino acid residue of SEQ ID NO: 25.

In one embodiment, the invention provides an isolated anti-GPC3 antibody or an antigen-binding portion thereof, comprising a heavy chain complementarity determining region 1 (H-CDR1) comprising the amino acid residue of SEQ ID NO: 4, a heavy chain CDR2 (H-CDR2) comprising the amino acid residue of SEQ ID NO: 8 and a heavy chain CDR3 (H-CDR3) comprising the amino acid residue of SEQ ID NO: 13; and a light chain CDR1 (L-CDR1) comprising the amino acid residue of SEQ ID NO: 15, a light chain CDR2 (L-CDR2) comprising the amino acid residue of SEQ ID NO: 21 and a light chain CDR3 (L-CDR3) comprising the amino acid residue of SEQ ID NO: 26.

In one embodiment, the invention provides an isolated anti-GPC3 antibody or an antigen-binding portion thereof, comprising a heavy chain complementarity determining region 1 (H-CDR1) comprising the amino acid residue of SEQ ID NO: 3, a heavy chain CDR2 (H-CDR2) comprising the amino acid residue of SEQ ID NO: 9 and a heavy chain CDR3 (H-CDR3) comprising the amino acid residue of SEQ ID NO: 14; and a light chain CDR1 (L-CDR1) comprising the amino acid residue of SEQ ID NO: 17, a light chain CDR2 (L-CDR2) comprising the amino acid residue of SEQ ID NO: 22 and a light chain CDR3 (L-CDR3) comprising the amino acid residue of SEQ ID NO: 27.

The amino acid sequences of the complementarity determining regions in heavy chains and light chains are listed below respectively.

### CDRs of Heavy Chain

| H-CDR1 | H-CDR2 | H-CDR3 |
|---|---|---|
| GFTFSSYA (SEQ ID | VSKDGTTT (SEQ ID NO: 5) | AKSNTNSRAAGLIDA (SEQ ID |
| NO: 1) | | NO: 10) |
| GFTFSSVN (SEQ ID | ISNTNTT (SEQ ID NO: 6) | ARGSGVSGTYAGQIDA (SEQ ID |
| NO: 2) | | NO: 11) |
| GFTFSSFN (SEQ ID | ISSTGSRT (SEQ ID NO: 7) | AKSASRGAGRIDA (SEQ ID NO: |
| NO: 3) | | 12) |
| GFTFNNYC (SEQ | ISKDGSTP (SEQ ID NO: 8) | ARGGGSNYCGSTGRINA (SEQ ID |
| ID NO: 4) | | NO: 13) |
| | ISGTGSST (SEQ ID NO: 9) | AKGVDSDSWTAAGIDA (SEQ ID |
| | | NO: 14) |

### CDRs of Light Chain

| L-CDR1 | L-CDR2 | L-CDR3 |
|---|---|---|
| SGSYG (SEQ ID NO: 15) | ANT (SEQ ID NO: 18) | GSRDSSYVGI (SEQ ID NO: 23) |
| GGSYG (SEQ ID NO: 16) | QND (SEQ ID NO: 19) | GNYDGNTDSGYVGV (SEQ ID |
| | | NO: 24) |
| TGRWYG (SEQ ID NO: 17) | NNN (SEQ ID NO: 20) | GSRDSSYFGI (SEQ ID NO: 25) |
| | RNN (SEQ ID NO: 21) | GNAGSSTYAGI (SEQ ID NO: 26) |
| | SND (SEQ ID NO: 22) | GSRESSRNPGI (SEQ ID NO: 27) |

According to the invention, the embodiments of the amino acids of the heavy chains and light chains of the antibodies of the invention are listed below.

Embodiments of Amino Acid Sequences of Heavy Chains

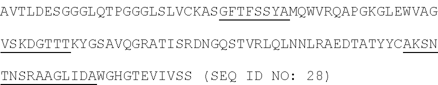

G5S1 (555 S1)

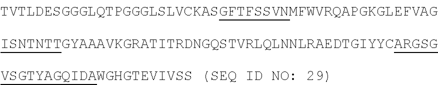

G5S8 (555 S8)

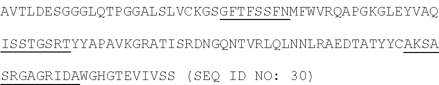

GES1 (GPC3 S1)

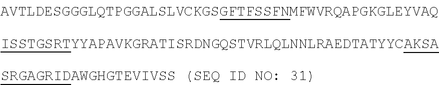

GES2 (GPC3 S2)

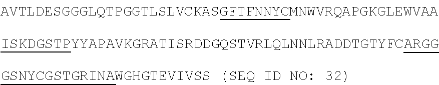

GES6 (GPC3 S6)

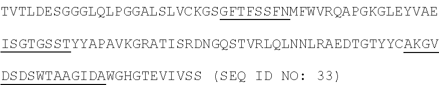

GES8 (GPC3 S8)

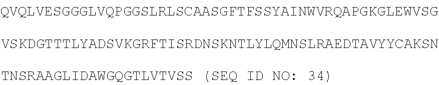

G5S1 humanized scFv sequence

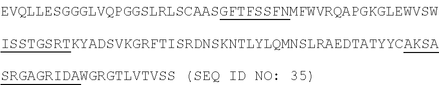

(GES1 humanized scFv sequence)

Embodiments of Amino Acid Sequences of Light chains

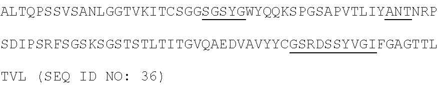

G5S1 (555 S1)

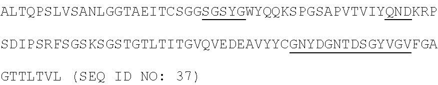

G5S8 (555 S8)

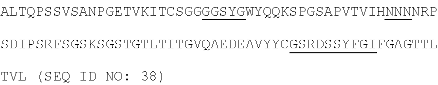

GES1 (GPC3 S1)

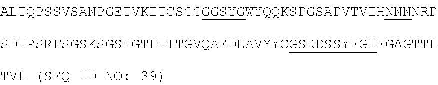

GES2 (GPC3 S2)

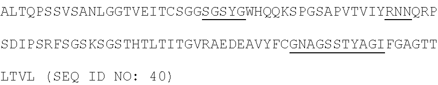

GES6 (GPC3 S6)

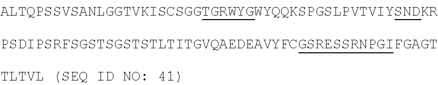

GES8 (GPC3 S8)

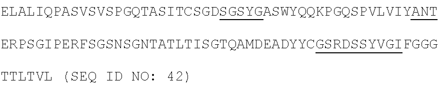

G5S1 humanized scFv sequence

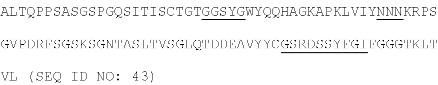

GES1 humanized scFv sequence

In some embodiments, the invention provides a heavy chain comprising an amino acid sequence having a sequence selected from the group consisting of as set forth in SEQ ID NOs: 28 to 35.

In some embodiments, the heavy chain comprises an amino acid sequence having the sequence as set forth in SEQ ID NO: 28 wherein the H-CDR1, H-CDR2 and H-CDR3 are replaced with any of SEQ ID NOs: 2 to 4, any of SEQ ID NOs: 6 to 9 and any of SEQ ID NOs: 11 to 14, respectively.

In some embodiments, the heavy chain comprises an amino acid sequence having the sequence as set forth in SEQ ID NO: 29 wherein the H-CDR1, H-CDR2 and H-CDR3 are replaced with any of SEQ ID NOs: 1, 3 and 4, any of SEQ ID NOs: 5, and 7 to 9 and any of SEQ ID NOs: 10 and 12 to 14, respectively.

In some embodiments, the heavy chain comprises an amino acid sequence having the sequence as set forth in SEQ ID NO: 30 wherein the H-CDR1, H-CDR2 and H-CDR3 are replaced with any of SEQ ID NOs: 1, 2 and 4, any of SEQ ID NOs: 5, 6, 8 and 9 and any of SEQ ID NOs: 10, 11, 13 and 14, respectively.

In some embodiments, the heavy chain comprises an amino acid sequence having the sequence as set forth in SEQ ID NO: 31 wherein the H-CDR1, H-CDR2 and H-CDR3 are replaced with any of SEQ ID NOs: 1, 2 and 4, any of SEQ ID NOs: 5, 6, 8 and 9 and any of SEQ ID NOs: 10, 11, 13 and 14, respectively.

In some embodiments, the heavy chain comprises an amino acid sequence having the sequence as set forth in SEQ ID NO: 32 wherein the H-CDR1, H-CDR2 and H-CDR3 are replaced with any of SEQ ID NOs: 1 to 3, any of SEQ ID NOs: 5 to 7 and 9 and any of SEQ ID NOs: 10 to 12 and 14, respectively.

In some embodiments, the heavy chain comprises an amino acid sequence having the sequence as set forth in SEQ ID NO: 33 wherein the H-CDR1, H-CDR2 and H-CDR3 are replaced with any of SEQ ID NOs: 1, 2 and 4, any of SEQ ID NOs: 5 to 8, and any of SEQ ID NOs: 10 to 13, respectively.

In some embodiments, the heavy chain comprises an amino acid sequence having the sequence as set forth in SEQ ID NO: 34, wherein the H-CDR1, H-CDR2 and H-CDR3 are replaced with any of SEQ ID NOs: 2 to 4, any of SEQ ID NOs: 6 to 9, and any of SEQ ID NOs: 11 to 14, respectively.

In some embodiments, the heavy chain comprises an amino acid sequence having the sequence as set forth in SEQ ID NO: 35, wherein the H-CDR1, H-CDR2 and H-CDR3 are replaced with any of SEQ ID NOs: 1, 2 and 4, any of SEQ ID NOs: 5, 6, 8 and 9, and any of SEQ ID NOs: 10, 11, 13 and 14, respectively.

In some embodiments, the invention provides a light chain comprising an amino acid sequence having the sequence selected from the group consisting of as set forth in SEQ ID NOs: 36 to 43.

In some embodiments, the light chain comprises an amino acid sequence having the sequence as set forth in SEQ ID NO: 36 wherein the L-CDR1, L-CDR2 and L-CDR3 are replaced with any of SEQ ID NOs: 16 and 17, any of SEQ ID NOs: 19 to 22 and any of SEQ ID NOs: 24 to 27, respectively.

In some embodiments, the light chain comprises an amino acid sequence having the sequence as set forth in SEQ ID NO: 37 wherein the L-CDR1, L-CDR2 and L-CDR3 are replaced with any of SEQ ID NOs: 16 and 17, any of SEQ ID NOs: 18 and 20 to 22 and any of SEQ ID NOs: 23 and 25 to 27, respectively.

In some embodiments, the light chain comprises an amino acid sequence having the sequence as set forth in SEQ ID NO: 38 wherein the L-CDR1, L-CDR2 and L-CDR3 are replaced with any of SEQ ID NOs: 15 and 17, any of SEQ ID NOs: 18, 19, 21 and 22 and any of SEQ ID NOs: 23, 24, 26 and 27, respectively.

In some embodiments, the light chain comprises an amino acid sequence having the sequence as set forth in SEQ ID NO: 39 wherein the L-CDR1, L-CDR2 and L-CDR3 are replaced with any of SEQ ID NOs: 15 or 17, any of SEQ ID NOs: 18, 19, 21 and 22 and any of SEQ ID NOs: 23, 24, 26 and 27, respectively.

In some embodiments, the light chain comprises an amino acid sequence having the sequence as set forth in SEQ ID NO: 40 wherein the L-CDR1, L-CDR2 and L-CDR3 are replaced with any of SEQ ID NOs: 16 and 17, any of SEQ ID NOs: 18, 19, 20 and 22 and any of SEQ ID NOs: 23 to 25 and 27, respectively.

In some embodiments, the light chain comprises an amino acid sequence having the sequence as set forth in SEQ ID NO: 41 wherein the L-CDR1, L-CDR2 and L-CDR3 are replaced with any of SEQ ID NOs: 15 and 16, any of SEQ ID NOs: 18 to 21 and any of SEQ ID NOs: 23 to 26, respectively.

In some embodiments, the light chain comprises an amino acid sequence having the sequence as set forth in SEQ ID NO: 42 wherein the L-CDR1, L-CDR2 and L-CDR3 are replaced with any of SEQ ID NOs: 16 and 17, any of SEQ ID NOs: 17 and 19-22 and any of SEQ ID NOs: 24 to 27, respectively.

In some embodiments, the light chain comprises an amino acid sequence having the sequence as set forth in SEQ ID NO: 43 wherein the L-CDR1, L-CDR2 and L-CDR3 are replaced with any of SEQ ID NOs: 15 and 17, any of SEQ ID NOs: 18, 19, 21 and 22 and any of SEQ ID NOs: 23, 24, 26 and 27, respectively.

In further embodiments, the invention comprises an isolated antibody, comprising a heavy chain having an amino acid sequence as set forth in the sequence selected from the group consisting of SEQ ID NOs: 28 to 35 or a variant having at least 80% identical to any of SEQ ID NOs: 28 to 35, and (ii) a light chain having an amino acid sequence as set forth in the sequence selected from the group consisting of SEQ ID NOs: 36 to 43 or a variant having at least 80% identical to any of SEQ ID NOs: 36 to 43. Preferably, the sequence identity as mentioned above is at least 90%, 91%, 91%, 92%, 93%, 94%, 95%, 96%, 97%, 98% or 99%.

In a further embodiment, the invention comprises an isolated antibody, comprising a heavy chain having an amino acid sequence as set forth in SEQ ID NO: 28 (G5S1 (555S1)) and a light chain having an amino acid sequence as set forth in SEQ ID NO: 36 (G5S1 (555S1)). In a further embodiment, the invention comprises an isolated antibody, comprising a heavy chain having an amino acid sequence as set forth in SEQ ID NO: 29 (G5S8 (555S8)) and a light chain having an amino acid sequence as set forth in SEQ ID NO: 37 (G5S8 (555S8)). In a further embodiment, the invention comprises an isolated antibody, comprising a heavy chain having an amino acid sequence as set forth in SEQ ID NO: 30 (GES1 (GPC3 S1)) and a light chain having an amino acid sequence as set forth in SEQ ID NO: 38 (G5S8 (555S8)). In a further embodiment, the invention comprises an isolated antibody, comprising a heavy chain having an amino acid sequence as set forth in SEQ ID NO: 31 (GES2 (GPC3 S2)) and a light chain having an amino acid sequence as set forth in SEQ ID NO: 39 (G5S8 (555S8)). In a further embodiment, the invention comprises an isolated antibody, comprising a heavy chain having an amino acid sequence as set forth in SEQ ID NO: 32 (GES6 (GPC3 S6)) and a light chain having an amino acid sequence as set forth in SEQ ID NO: 40 (G5S8 (555S8)). In a further embodiment, the invention comprises an isolated antibody, comprising a heavy chain having an amino acid sequence as set forth in SEQ ID NO: 33 (GES8 (GPC3 S8)) and a light chain having an amino acid sequence as set forth in SEQ ID NO: 41 (G5S8 (555S8)).

In another aspect, the present invention provides variants of an antagonist antibody or fragment thereof that binds to GPC3. Thus the present invention provides antibodies or fragments thereof that have an amino acid sequence of the non-CDR regions of the heavy and/or light chain variable region sequence which is at least 80% identical (having at least 80% amino acid sequence identity) to the amino acid sequence of the non-CDR regions of the heavy and/or light chain variable region sequence of the parent antagonist antibody of either the heavy or the light chain. Preferably the amino acid sequence identity of the non-CDR regions of the heavy and/or light chain variable region sequence is at least 85%, more preferably at least 90%, and most preferably at least 95%, in particular 96%, more particularly 97%, even more particularly 98%, most particularly 99%, including for example, 80%, 81%, 82%, 83%, 84%, 85%, 86%, 87%, 88%, 89%, 90%, 91%, 92%, 93%, 94%, 95%, 96%, 97%, 98%, 99%, and 100%. Identity or homology with respect to an amino acid sequence is defined herein as the percentage of amino acid residues in the candidate sequence that are identical with the antagonist antibody or fragment thereof that binds to GPC3, after aligning the sequences and introducing gaps, if necessary, to achieve the maximum percent sequence identity. Thus, sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM250 (a standard scoring matrix; see Dayhoff M O et al., (1978) in Atlas of Protein Sequence and Structure, vol 5, supp. 3) can be used in conjunction with the computer program. For example, the percent identity can be calculated as: the total number of identical matches multiplied by 100 and then divided by the sum of the length of the longer sequence within the matched span and the number of gaps introduced into the longer sequences in order to align the two sequences.

In another aspect, the present disclosure also provides an antibody or fragment thereof that binds to GPC3, wherein at least one of the heavy chain CDRs and/or at least one of the light chain CDRs comprises at least one amino acid modification. Site-directed mutagenesis or PCR-mediated mutagenesis can be performed to introduce the modification(s) and the effect on antibody binding, or other functional property of interest, can be evaluated in in vitro or in vivo assays. Preferably conservative modifications are introduced. The modifications may be amino acid substitutions, additions or deletions, but are preferably substitutions. Typically, no more than five, preferably no more than four, more preferably no more than three, even more preferably no more than two, most preferably no more than one amino acid modification is performed within a CDR region.

In certain embodiments, framework sequences can be used to engineer variable regions to produce variant antibodies. Variant antibodies of the invention include those in which modifications have been made to framework residues within VH and/or VK, e.g. to improve the properties of the antibody. Typically, such framework modifications are made to decrease the immunogenicity of the antibody. For example, one approach is to “backmutate” one or more framework residues to the corresponding murine sequence or to “backmutate” one or more framework residues to a corresponding germline sequence.

In some embodiments, the isolated anti-GPC3 antibody is a monoclonal antibody, chimeric antibody, humanized antibody or human antibody. The present disclosure also provides a monovalent antibody or fragment thereof that binds to GPC3, i.e. an antibody which consists of a single antigen binding arm. The present disclosure also provides a fragment of an antibody that binds to GPC3 selected from the group consisting of Fab, Fab′, Fab′-SH, Fd, Fv, dAb, F(ab′)2, scFv, bispecific single chain Fv dimers, diabodies, triabodies and scFv genetically fused to the same or a different antibody. Preferred fragments are scFv, bispecific single chain Fv dimers and diabodies. The present disclosure also provides a full length antibody that binds to GPC3.

Techniques for preparing monoclonal antibodies against virtually any target antigen are well known in the art. See, for example, Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991). Briefly, monoclonal antibodies can be obtained by injecting mice or chicken with a composition comprising an antigen, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

Various techniques, such as production of chimeric or humanized antibodies, may involve procedures of antibody cloning and construction. The antigen-binding variable light chain and variable heavy chain sequences for an antibody of interest may be obtained by a variety of molecular cloning procedures, such as RT-PCR, 5′-RACE, and cDNA library screening. The variable heavy or light chain sequence genes of an antibody from a cell that expresses a murine antibody can be cloned by PCR amplification and sequenced. To confirm their authenticity, the cloned V_{L} and V_{H} genes can be expressed in cell culture as a chimeric antibody as described by Orlandi et al., (Proc. Natl. Acad. Sci., USA, 86: 3833 (1989)). Based on the variable heavy or light chain gene sequences, a humanized antibody can then be designed and constructed as described by Leung et al. (Mol. Immunol., 32: 1413 (1995)).

A chimeric antibody is a recombinant protein in which the variable regions of a human antibody have been replaced by the variable regions of, for example, a mouse antibody, including the complementarity-determining regions (CDRs) of the mouse antibody. Chimeric antibodies exhibit decreased immunogenicity and increased stability when administered to a subject. Methods for constructing chimeric antibodies are well known in the art (e.g., Leung et al., 1994, Hybridoma 13:469).

A chimeric monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. To preserve the stability and antigen specificity of the humanized monoclonal, one or more human FR residues may be replaced by the mouse counterpart residues. Humanized monoclonal antibodies may be used for therapeutic treatment of subjects. Techniques for production of humanized monoclonal antibodies are well known in the art. (See, e.g., Jones et al., 1986, Nature, 321:522; Riechmann et al., Nature, 1988, 332:323; Verhoeyen et al., 1988, Science, 239:1534; Carter et al., 1992, Proc. Nat'l Acad. Sci. USA, 89:4285; Sandhu, Crit. Rev. Biotech., 1992, 12:437; Tempest et al., 1991, Biotechnology 9:266; Singer et al., J. Immun., 1993, 150:2844.

In one embodiment, the antibody is humanized scFv antibody. In a further embodiment, the humanized scFv antibody comprises a heavy chain having an amino acid sequence as set forth in SEQ ID NO: 34 and a light chain having an amino acid sequence as set forth in SEQ ID NO: 42 (G5S1 humanized scFv antibody). In another further embodiment, the invention comprises a humanized scFv antibody, comprising a heavy chain having an amino acid sequence as set forth in SEQ ID NO: 35 and a light chain having an amino acid sequence as set forth in SEQ ID NO: 43 (GES1 humanized scFv antibody).

Modifications can be made to a nucleic acid encoding a polypeptide described herein without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps. In addition to recombinant methods, the antibodies of the present disclosure can also be constructed in whole or in part using standard peptide synthesis well known in the art.

As a modification to the two chain antibody purification protocol, the heavy and light chain regions are separately solubilized and reduced and then combined in the refolding solution. An exemplary yield is obtained when these two proteins are mixed in a molar ratio such that a 5-fold molar excess of one protein over the other is not exceeded. Excess oxidized glutathione or other oxidizing low molecular weight compounds can be added to the refolding solution after the redox-shuffling is completed.

In addition to recombinant methods, the antibodies and variants thereof that are disclosed herein can also be constructed in whole or in part using standard peptide synthesis. Solid phase synthesis of the polypeptides can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chem. Soc. 85:2149-2156, 1963, and Stewart et al., Solid Phase Peptide Synthesis, 2nd ed., Pierce Chem. Co., Rockford, Ill., 1984. Proteins of greater length may be synthesized by condensation of the amino and carboxyl termini of shorter fragments.

### Anti-GPC3 Antibody Compositions and Methods of Administrations

Certain embodiments relate to a pharmaceutical composition comprising the anti-GPC3 antibody of the invention and a pharmaceutically acceptable carrier or excipient. By “pharmaceutically acceptable carrier” is intended, but not limited to, a non-toxic solid, semisolid or liquid filler, diluent, encapsulating material or formulation auxiliary of any type known to persons skilled in the art. Diluents, such as polyols, polyethylene glycol and dextrans, may be used to increase the biological half-life of the conjugate.

The pharmaceutical compositions of the present invention can be formulated according to conventional methods (for example, Remington's Pharmaceutical Science, latest edition, Mark Publishing Company, Easton, U.S.A.), and may also contain pharmaceutically acceptable carriers and additives. Examples include, but are not limited to, surfactants, excipients, coloring agents, flavoring agents, preservatives, stabilizers, buffers, suspension agents, isotonic agents, binders, disintegrants, lubricants, fluidity promoting agents, and corrigents, and other commonly used carriers can be suitably used. Specific examples of the carriers include light anhydrous silicic acid, lactose, crystalline cellulose, mannitol, starch, carmellose calcium, carmellose sodium, hydroxypropyl cellulose, hydroxypropyl methylcellulose, polyvinylacetal diethylaminoacetate, polyvinylpyrrolidone, gelatin, medium-chain triglyceride, polyoxyethylene hardened castor oil 60, saccharose, carboxymethyl cellulose, corn starch, inorganic salt, and such.

Certain embodiments are directed to a method for treating a cancer in a subject comprising administering an anti-GPC3 antibody of the invention to the subject. The invention also provides a use of an anti-GPC3 of the invention in the manufacture of a medicament for treating a cancer. The present method also comprises administering the anti-GPC3 antibody of the invention concomitantly with, or subsequent to other standard therapies, wherein said standard therapy is selected from the group consisting of radiotherapy, surgery and chemotherapy.

In preferred embodiments, the subject is a mammal. Exemplary mammals include human, pig, sheep, goat, horse, mouse, dog, cat, cow, etc. Diseases that may be treated with the anti-GPC3 antibody or a pharmaceutical composition thereof include cancer, such as cancer of the liver, skin, head and neck, lung, breast, prostate, ovaries, endometrium, cervix, colon, rectum, bladder, brain, stomach, pancreas or lymphatic system. Preferably, the cancer is liver cancer such as hepatocellular carcinoma (HCC), hepatoblastoma and sarcomatoid HCC.

The anti-GPC3 antibody or the pharmaceutical composition thereof may be administered intravenously, intra-peritoneally, intra-arterially, intra-thecally, intra-vesically, or intratumorally. One of ordinary skill will appreciate that effective amounts of the anti-GPC3 antibody can be determined empirically. It will be understood that, when administered to a human patient, the total daily usage of the anti-GPC3 antibody or composition will be decided by the attending physician within the scope of sound medical judgment. The specific therapeutically effective dose level for any particular patient will depend upon a variety of factors: the type and degree of the cellular response to be achieved; activity of the specific anti-GPC3 antibody or composition employed; the age, body weight, general health, sex and diet of the patient; the time of administration, route of administration, and rate of excretion of the anti-GPC3 antibody; the duration of the treatment; drugs used in combination or coincidental with the anti-GPC3 antibody; and like factors well known in the medical arts.

Each of the above identified compositions and methods of treatment may additionally include an additional anti-tumor drug and the administration of an additional one or more anti-tumor drug. Anti-tumor drugs suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like. Examples of the additional anti-tumor drug includes but is not limited to 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxins, and/or radionuclides, etc.; 9) biological response modifiers (e.g., interferons (e.g., IFN-.alpha., etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); and 22) modulators of p53 protein function.

### Diagnosis of Cancer by Expression of GPC3

The present invention surprisingly found that highly expression of GPC3 is associated with a cancer. Accordingly, the invention provides a method for diagnosing a cancer in a subject, comprising detecting a binding of the antibody of the invention to GPC3 in a biological sample, wherein the binding indicates likelihood of the subject developing a cancer. The cancer includes, but is not limited to, ovarian cancer, breast cancer, liver cancer, lung cancer, non-small cell lung cancer, small cell lung cancer (including small cell carcinoma (oat cell cancer), mixed small cell/large cell carcinoma, and combined small cell carcinoma), colon cancer, prostate cancer, pancreatic cancer, brain cancer, kidney cancer, stomach cancer, melanoma, bone cancer, gastric cancer, breast cancer, glioma, gliobastoma, hepatocellular carcinoma, papillary renal carcinoma, head and neck squamous cell carcinoma, leukemia, lymphoma, myeloma, or other tumors. Particularly, highly expression of GPC3 could enrich HCC-related genes' mRNA expression and positive associate with dysplasia in cirrhotic livers; therefore, GPC3 may serve as a precancerous biomarker in cirrhotic livers and liver cancers. Moreover, increase of HCC-related genes' mRNA expression by GPC3 was confirmed significantly in cirrhotic live. Therefore, GPC3 is useful as a specific marker for detecting a cirrhotic liver or liver cancer. Accordingly, in another aspect, the present invention provides a method for diagnosing a cirrhotic liver or liver cancer in a subject, comprising detecting a binding of the antibody of the invention to GPC3 in a biological sample, wherein the binding indicates that the subject is in the likelihood of developing a cirrhotic liver and liver cancer.

Biological samples used in the diagnosis methods of the present invention are not particularly limited as long as they are samples that may contain a GPC3 protein. Specifically, samples collected from the body of an organism such as mammal are preferred. Samples collected from humans are more preferred. Specific examples of the test samples include blood, interstitial fluid, plasma, extravascular fluid, cerebrospinal fluid, synovial fluid, pleural fluid, serum, lymphatic fluid, saliva, urine, tissue, ascites, and intraperitoneal lavage.

Methods for detecting the binding of the anti-GPC3 antibody of the invention to GPC3 protein contained in a test sample are not particularly limited. An immunological method using an anti-GPC3 antibody for detection such as radioimmunoassay (RIA); enzyme immunoassay (EIA); fluorescence immunoassay (FIA); luminescence immunoassay (LIA); immunoprecipitation (IP); turbidimetric immunoassay (TIA); Western blotting (WB); immunohistochemical (IHC) method; and single radial immunodiffusion (SRID).

The present invention also provides diagnostic agents or kits for diagnosing a cancer, comprising a diagnostic agent for detecting the GPC3 protein in a test sample. In one embodiment, the present invention also provides diagnostic agents or kits for diagnosing a cirrhotic liver or liver cancer, comprising a diagnostic agent for detecting the GPC3 protein in a test sample. The diagnostic agents of the present invention comprise at least an anti-GPC3 antibody of the invention.

Kits for diagnosing cancer can be produced by combining the agents for diagnosing a cirrhotic liver or liver cancer with another element used for detecting GPC3. More specifically, the present invention relates to kits for diagnosing a cirrhotic liver or liver cancer which comprise an anti-GPC3 antibody that binds to GPC3 and a reagent for detecting binding between the antibody and GPC3. In addition, instructions that describe the measurement operation can be attached to the kits of the present invention.

The present invention suggests that the functional domain or antigenic epitopes present in GPC3 protein may serve as a potential target for diagnostic or therapeutic application clinically. Accordingly, the present invention provides anti-GPC3 antibodies having anti-tumor activities and their applications for diagnostic and therapeutic purposes including the inhibition of tumor growth, proliferation and migration.

### EXAMPLES

### Example 1 GPC3 Protein Expression and Purification and Construction of scFv Antibody Libraries and Biopanning

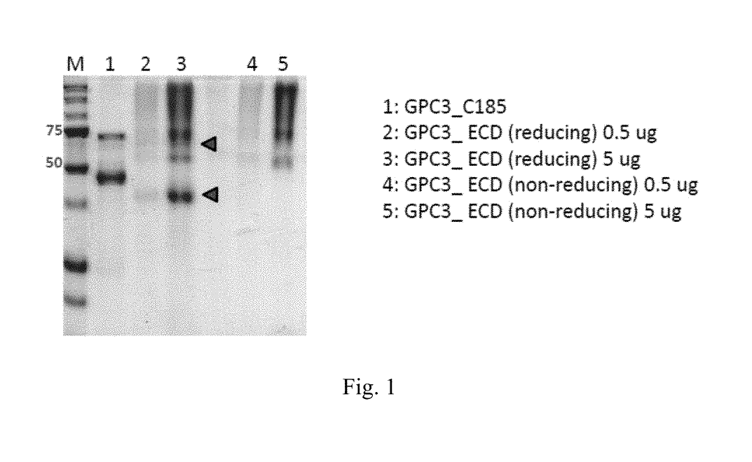

The various fragments of genes encoding human GPC3 protein were amplified by PCR, cloned into the pET21a vector, and transformed into the E. coli BL-21 (DE3) strain for expression as His-fused GPC3. Individual clones were grown in 5 ml LB medium containing ampicillin (100 μg/ml) at 37° C. overnight. The bacterial culture was diluted 10-fold in the same LB medium and further grown until the OD600 reached between 0.6 and 1.0. Isopropyl-β-D-thiogalactopyranoside (IPTG) was added to a final concentration of 0.5 mM in the culture. The cell pellet was re-suspended in 2 ml of 1×PBS containing 1% Triton x-100 and lysed by sonication in the presence of protease inhibitors. After centrifugation, the resulting cellular lysate was incubated with a Ni₂₊-charged resin column to purify the GPC3 protein according to the manufacturer's instruction (General Electronics, Piscataway, N.J., USA). FIG. 1 shows the results of analysis of commercial GPC3_extracellular domain (ECD) and truncated (C185) proteins.

Female white leghorn (Gallus domesticus) chickens were immunized with 100 ug of purified GPC3 in an equal volume of Freund's complete adjuvant by an intramuscular injection. Three additional immunizations with incomplete adjuvant were performed at intervals of 7 days. After each immunization, chicken IgY antibodies in egg yolk were collected and titrated by an enzyme-linked immunosorbent assay (ELISA) to determine the presence of humoral anti-GPC3 antibody immune response. The egg yolk will be separated from the egg white for IgY purification using 10% Dextran sulphate according to published protocol (Akita, E. M., and Nakai, S. (1993). Production and purification of Fab′ fragments from chicken egg yolk immunoglobulin Y (IgY). J Immunol Methods 162, 155-164).

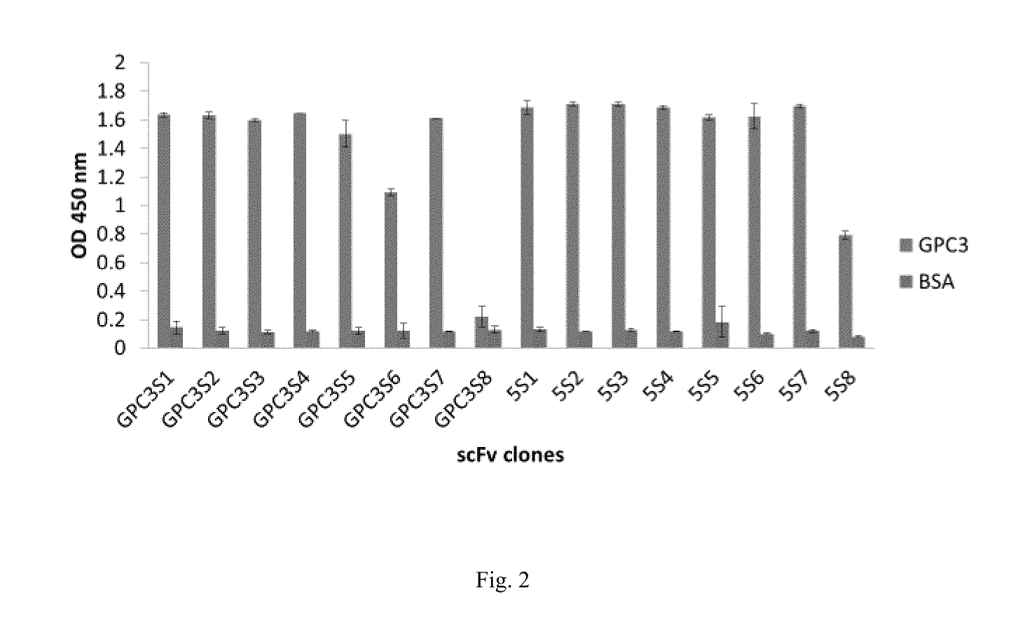

The antibody libraries will be established based on the previous report (Andris-Widhopf J., Rader, C., Steinberger, P., Fuller, R., and Barbas, C. F., 3rd (2000). Methods for the generation of chicken monoclonal antibody fragments by phage display. J Immunol Methods 242, 159-181. Barbas, C. F., 3rd, Kang, A. S., Lerner, R. A., and Benkovic, S. J. (1991). Assembly of combinatorial antibody libraries on phage surfaces: the gene III site. Proc Natl Acad Sci USA 88, 7978-7982). Briefly, chicken spleens were harvested and placed immediately in Trizol. Ten ug of total RNAs will be reversely transcribed into the first-strand cDNA. After amplification using chicken-specific primers, PCR products of heavy and light chain variable (VH and VL) regions with short or long linkers will be subjected to a second round of PCR, digested with SfiI and cloned into the pComb3× vector. Recombinant DNAs were transformed into E. coli ER2738 strain by electroporation. Recombinant phage was produced by the addition of VCS-M13 helper phage, precipitated with 4% polyethylglycol 8000 and 3% NaCl (w/v), re-suspended in phosphate-buffered saline (PBS) containing 1% bovine serum albumin (BSA). Then, 1011 plaque-forming units (pfu) of recombinant phages were added to wells pre-coated with GPC3 protein (0.5 ug/well), and incubated at 37° C. for 2 h. Bound phages were eluted with 0.1 M HCl/glycine (pH 2.2)/0.1% BSA, neutralized with 2 M Tris base buffer and used to infect the ER2738 strain. The amplified phages were recovered as described above for the next round of selection. The panning procedure was repeated three or four times. The total DNAs were purified and transformed into TOP 10F′ E. coli strain. Twenty clones were randomly selected and grown from the final panning process. Bacterial cells were lysed and analyzed for scFv antibody expression and binding reactivity to GPC3. ScFv antibodies were purified using Ni₂₊-charged sepharose as described by the manufacturer (Amersham Biosciences, UK). FIG. 2 shows the binding activity of anti-GPC3 antibodies using ELISA. The total cell lysates of 16 clones randomly selected from each ELISA-positive phage library after the 4^{th} round of bio-panning were used to examine their anti-GPC3 activity. The sequence analysis of heavy and light variable fragments used by these scFv antibodies suggested the identical gene usage were applied in GES2, GES3 and GES4 clones (data not shown). The nomenclature of GPC3S1-S8 and 5S1-S8 were replaced as GES1-S8 and G5S1-S8, respectively.

### Example 2 Purification of cscFv Against GPC3 by ELISA

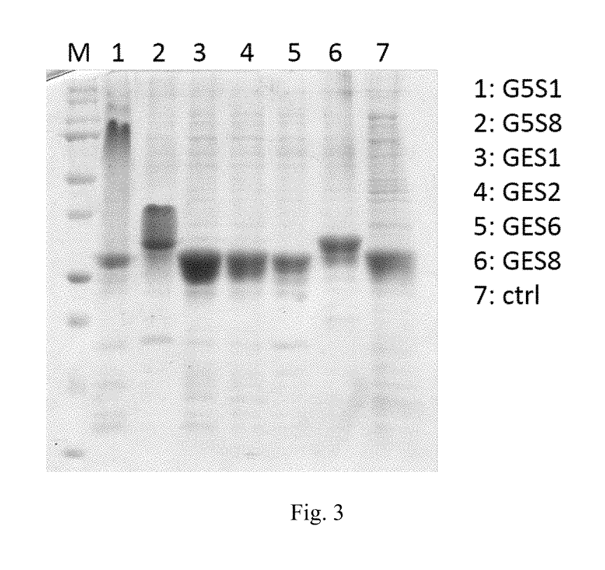

The scFv antibody expression was analyzed on sodium dodecylsulfate polyacrylamide gel electrophoresis (SDS-PAGE). Six clones with specific anti-GPC3 scFv were found to have satisfactory expression levels in the cytoplasm. FIG. 3 shows analysis of purified scFv antibodies on SDS-PAGE. scFv antibodies expressed in 6 clones, namely G5S1, G5S8, GES1, GES2, GES6 and GES8 (lanes 1-6), were purified and visualized by Coomassie blue staining. A scFv antibody (ctrl) reacting with snake venom protein was included as a control, showing a molecular weight of 25 kD. An extra band with a molecular weight larger than 25 kD in lanes 1 and 2 were believed to be the aggregated form (tetramer or dimer) of scFv antibodies since both anti-his and anti-chicken antibodies were able to react with these bands (not shown).

### Example 3 Sequence Alignment and Analysis of Anti-GPC3 cscFv

Conversion of scFv to Chicken-Human Chimeric Antibody

The VL and VH genes of anti-GPC3 scFv antibodies will be converted to exons by the recombinant PCR method (Tsurushita et al., 2004). At the first-step PCR, the signal peptide-coding region of the VL (or VH) will be amplified by PCR in such a way that the 5′ end contained an Kpn I site and the 3′ end will be attached to a sequence homologous to the 5′ end of the anti-GPC3 scFv VL (or VH) coding region (left-side fragment). The VL (or VH) of the scFv antibody will be amplified by PCR in such a way that the 5′ end will be attached to a sequence homologous to the 3′ end of the signal peptide-coding region of VL (or VH) while the 3′ end carried a splicing donor signal and an Nhe I site (right-side fragment). The left- and right-side fragments for each of anti-GPC3 scFv VL and VH will be combined and amplified by PCR to make a mini-exon flanked by Kpn I and Nhe I sites. After experimental validation, selected scFvs will be converted to IgG form for in vivo tests. The VH and VL genes of mouse/chicken scFv will be grafted into a human IgG scaffold to generate chimeric IgG constructions. IgG expression vector contains optimized constant domains for the heavy and light chains of the IgG1 immunoglobulin.

### Humanization

Humanization of variable regions of chicken scFv antibodies will be performed with the assistance of a molecular model generated by the algorithms applied in previous studies (Tsurushita et al., 2004; Zilber et al., 1990). In brief, the human V region framework used as an acceptor for the CDRs of the anti-GPC3 scFv antibodies will be chosen based on sequence homology. Amino acid residues in the humanized V regions predicted from the three-dimensional model to be important for proper formation of the CDR structure will be substituted with the corresponding residues of chicken anti-GPC3 scFv antibodies. Other methods will be combined and used to further fine-tune the structure of humanized anti-GPC3 antibodies (Ewert et al., 2003; Sidhu et al., 2004).

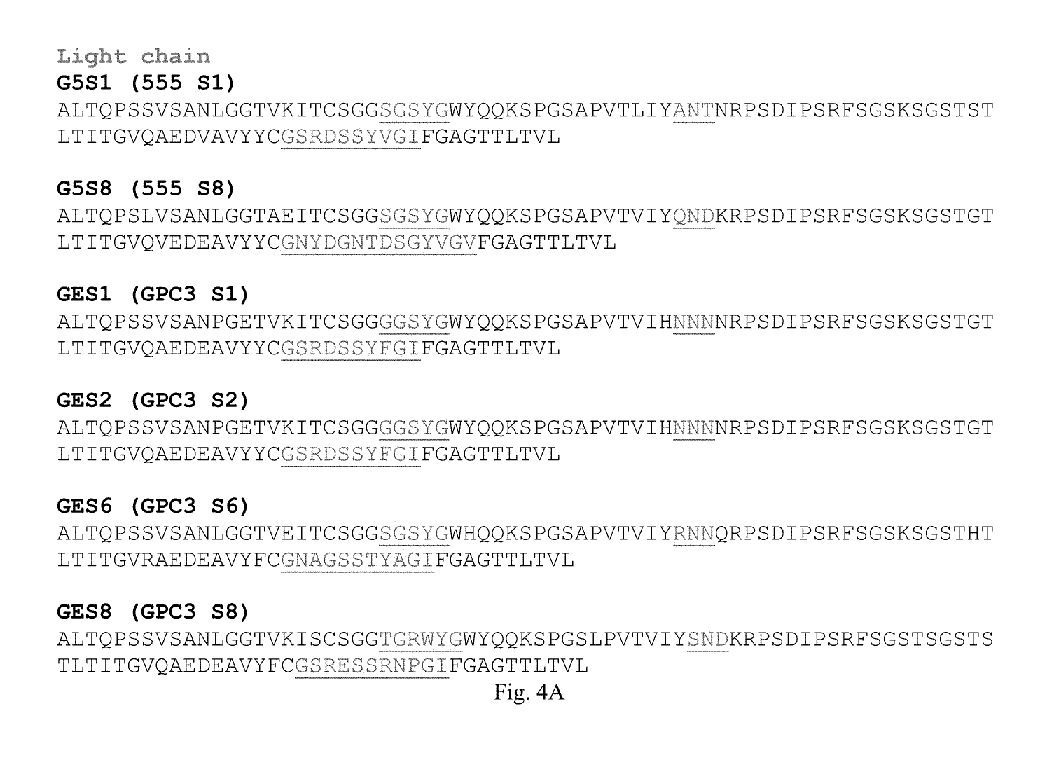

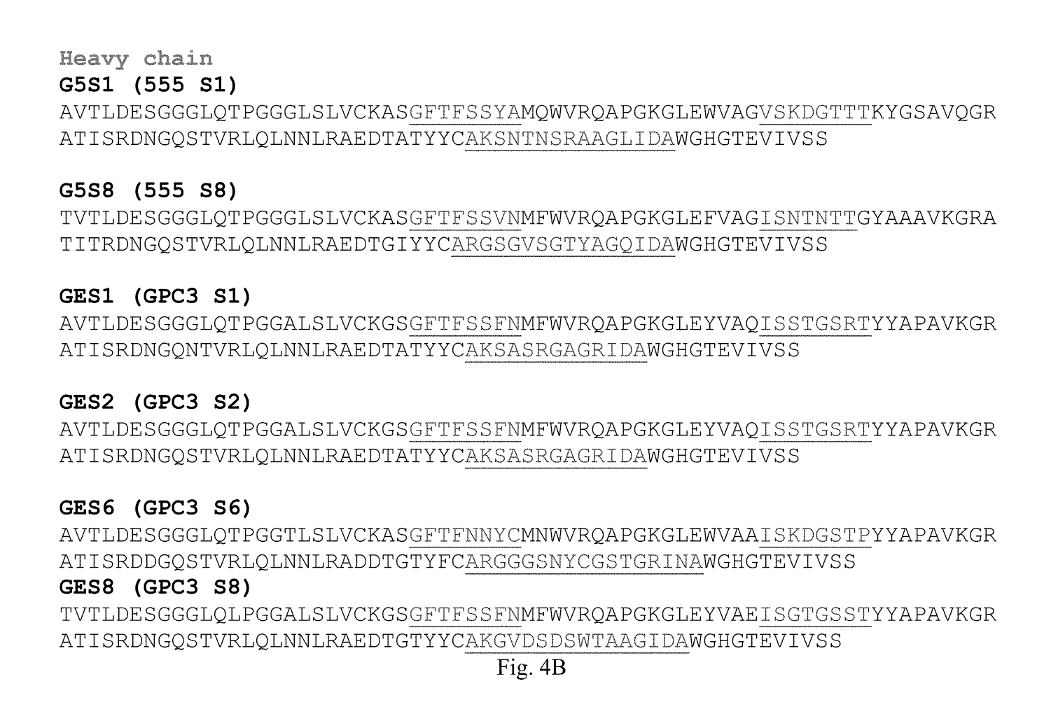

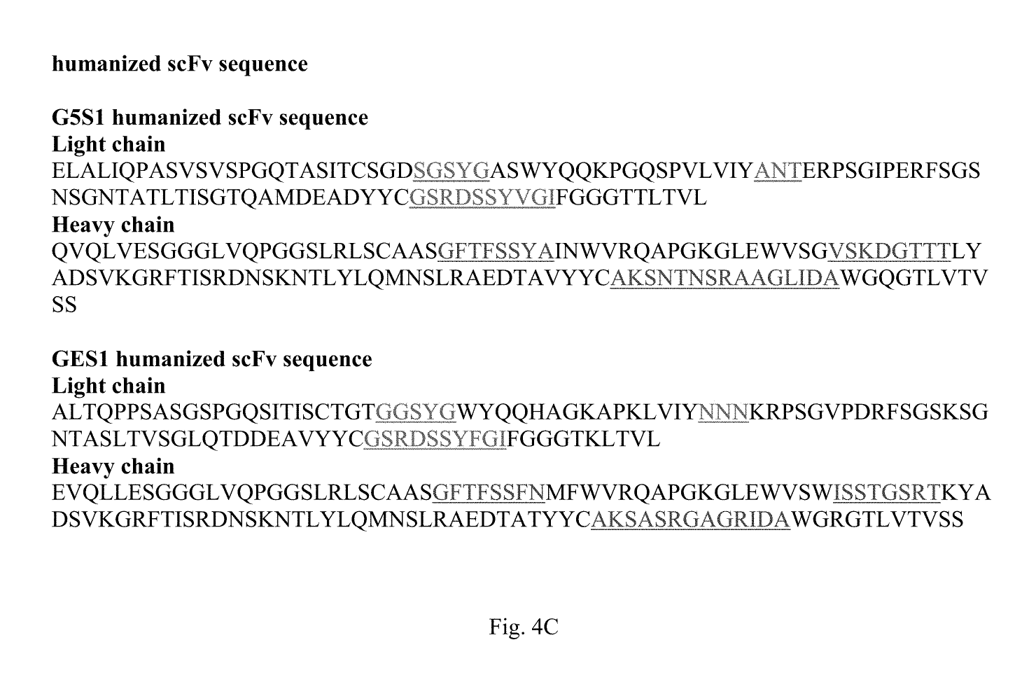

The nucleotide sequence determination of heavy and light variable regions from chosen scFv-expressing clones will be carried out by an auto-sequencer using ompseq (5′-AAGACAGCTATCGCGATTGCAGTG-3′) and HRML-F (5′-GGTGGTTCCTCTAGATCTTCC-3′) primers. The results will be analyzed using alignment program BLAST and Vector NTI (http://www.ncbi.nlm.nih.gov/BLAST). FIG. 4 shows the heavy chains (A) and light chains (B) of the selected scFv sequences of GPC3 gene of chicken (555 S1, S8 and GPC3 S1, S2, S6, S8) and heavy chains and light chains of humanized scFv sequence of the invention (G5S1 humanized scFv sequence and GES1 humanized scFv sequence).

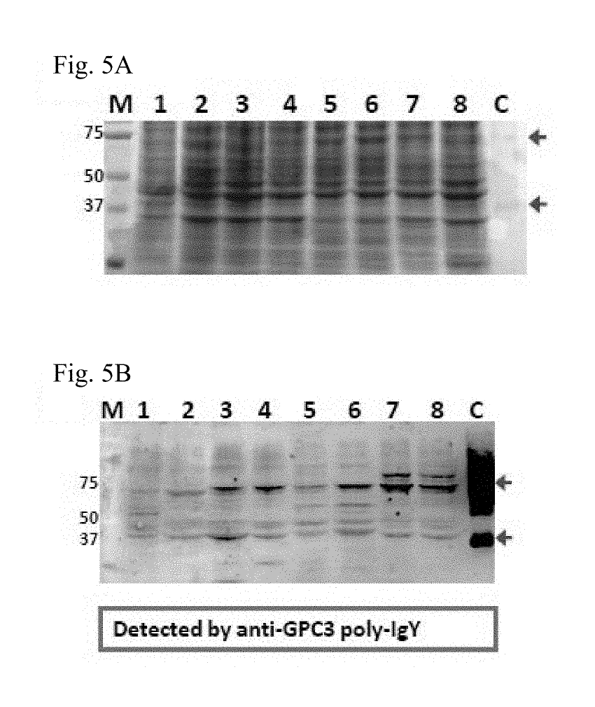

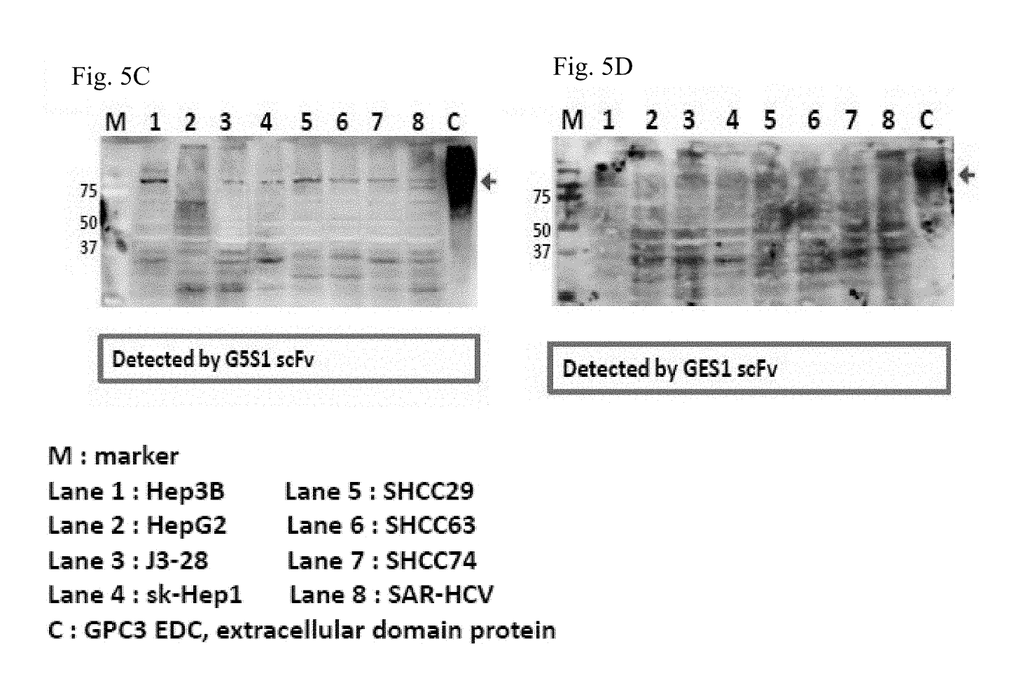

Nitrocellulose membranes were blocked with 5% skim milk in TBST for 1 hr. Polyclonal goat anti-chicken IgY light chain antibodies were added at 1:5000 dilution and incubated for an additional hr. The membranes were washed with TBST three times and the bound antibodies were detected by adding horseradish peroxidase (HRP)-conjugated donkey anti-goat Ig antibodies at 1:3000 dilutions. After three washings, the membranes were developed with diaminobenzidine (DAB) substrate until the desired intensity was reached. The IgY antibodies or the E. coli-expressed scFv antibodies were purified and incubated with the purified GPC3 protein or cellular lysates of GPC3-positive HepG2 and Hep3B cells immobilized on nitrocellulose membranes or ELISA plate wells. Their binding was subsequently detected by adding goat anti-mouse IgG or anti-chicken IgY light chain followed by HRP-conjugated donkey anti-goat Ig antibodies as described above. FIG. 5 shows electrophoresis analysis of cell lysates of four hepatoma cell lines (Lanes 1-4) and four sarcomatoid hepatoma cell lines (Lanes 5-8). FIG. 5A shows 8 cell lysates protein and commercial GPC3 extracellular domain protein under reducing condition (Lane C) and the upper arrow shows C-terminus fragment and the lower arrow shows N-terminus fragment. FIG. 5B shows two fragments identified by anti-GPC3 poly IgY. FIG. 5C shows the fragment identified by G5S1 scFv. FIG. 5D shows the fragment identified by GES1 scFv. The two scFvs of FIGS. 5C and D can identify C-terminus fragment of GPC3 and GPC3 in cellular lysates (see arrow).

### Example 4 Binding Analysis of Anti-GPC3 scFv Antibodies on ELISA

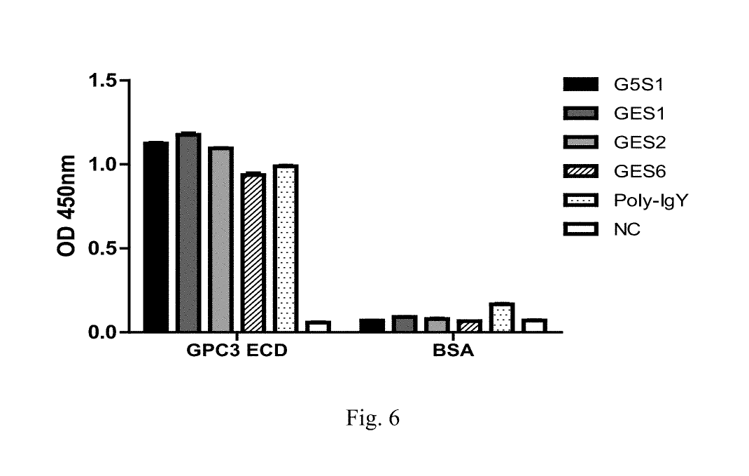

HepG2, Hep3B, cells (2×10⁵ cells/ml) was seeded on cover glass and fixed by incubating with equal volume of 8% freshly prepared ice-cold paraformaldehyde on ice for 15 min. After fixing, the cells were dehydrated in a sequential treatment of 70%, 95% and 99% methanol and rehydrated with 95% and 70% methanol. The slides were then overlaid with blocking buffer (1% BSA in 1×PBS) at room temperature (RT) for 1 hr. Following washing with 1×PBS, specific monoclonal mouse or scFv antibodies was incubated with cells at RT for one additional hr. Finally, their binding to GPC3 protein was detected by mouse anti-HA antibodies, followed by FITC-conjugated goat anti-mouse antibodies. Nuclei were counterstained with PI solution in parallel. The slides were examined using a Confocal Spectral Microscope Imaging System (TCS SP5, Leica). FIG. 6 shows the results of the binding analysis of specific anti-GPC3 scFv antibodies on ELISA. The partially purified scFv antibodies shown in FIG. 5 were examined for their binding activity to commercially available GPC3 immobilized on plate wells. The results showed that G5S1, GES1, GES2 and GES6 scFv antibodies reacted significantly with GPC3. Anti-snake venom scFv (NC) and polyclonal anti-GPC3 antibodies were included as negative and positive controls, respectively.

### Example 5 Proliferation (MTT) Assay of the Anti-GPC scFv Antibodies

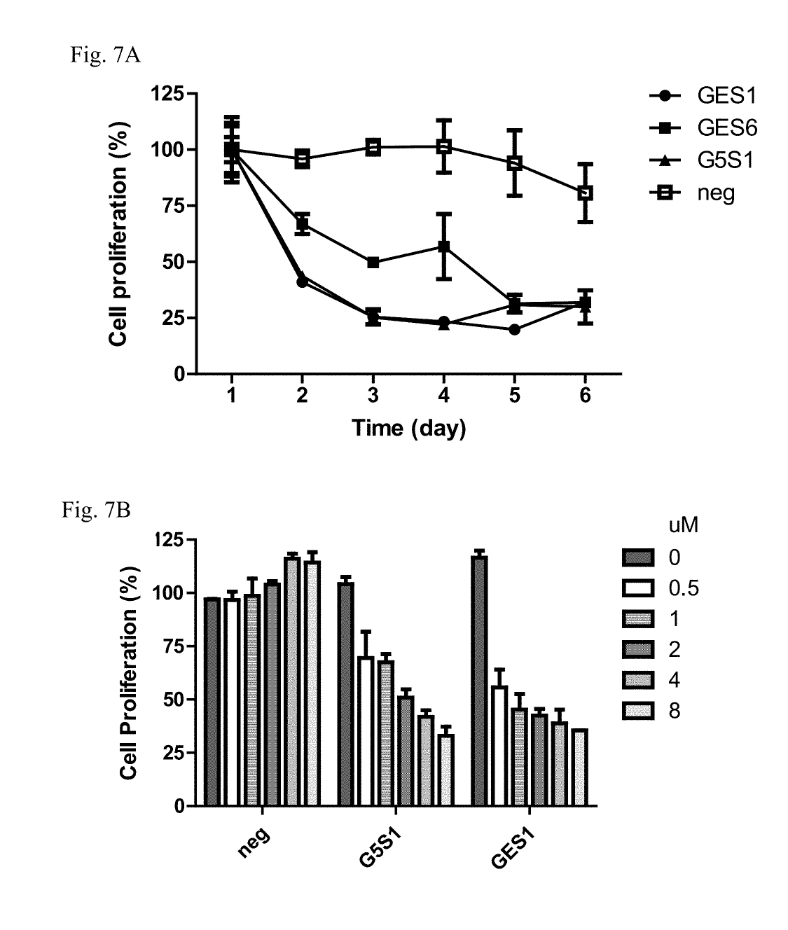

A volume of 100 μl DMEM medium containing 5×10³ HepG2, Hep3B, wherein cells were seeded onto 96-well plates. After 24 hr, the medium was replaced by fresh DMEM containing the anti-GPC3 scFv antibodies at final concentrations of 0.5-5 uM. The cells were cultured for 6 days. Thereafter, 10 μl of 3-[4,5-dimethylthiazol-2-yl]-2,5-diphenyltetrazolium bromide (MTT; 10 mg/ml) were added to determine the number of surviving cells. The supernatant was removed after 3 hr of incubation, and formazan crystals that had been developed were dissolved by adding of 100 μl acidified 2-propanol/well (0.04 N HCl). Extinction was measured by an automated microtiter plate reader at 540 nm. Unrelated anti-snake venom antibodies (neg) were included in control experiments. FIG. 7 shows that proliferative inhibition of specific anti-GPC3 scFv antibodies on hepatoma cells. HepG2 cells were cultured in the medium containing G5S1, GES1, GES6 or control scFv antibodies at final concentrations of 0.1 μM for 1-6 days. As seen in upper panel, the data indicated that 0.1 μM of G5S1, GES1 and GES6 exhibited approximately 60% of suppression on cell proliferation at days 6. The data in the low panel indicated that G5S1 and GES1 scFv antibodies at final concentrations of 0, 0.5, 1, 2, 4, and 8 μM for 3 days inhibited the cell proliferation in a dosage-dependent manner.

### Example 6 Flow Cytometry Analysis of the Anti-GPC scFv Antibodies

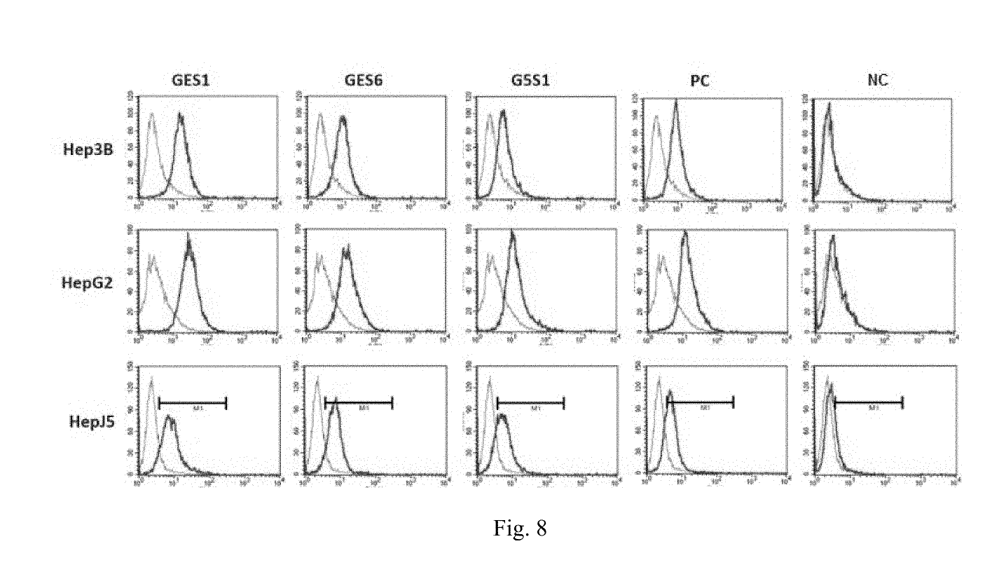

A total of 2×10⁶ HepG2, Hep3B, cells were harvested and fixed with 2% paraformaldehyde as described previously with minor modification (Leu et al., 2010). The GPC3 molecule expressed in these canceqazer cells were detected by adding purified chicken scFv antibodies, followed by mouse anti-HA (1:200) and FITC-conjugated goat anti-mouse antibodies (1:200). The results were analyzed using the FACScan flow cytometer Negative controls were performed by omitting the primary scFv antibodies, while positive controls were performed using commercial polyclonal anti-human GPC3 antibodies (1:200) instead of home-made chicken anti-GPC3 scFv antibodies. FIG. 8 shows the binding analysis of specific anti-GPC3 scFv antibodies using flow cytometry. The partially purified scFv antibodies were examined for their binding activity to GPC3-positive Hep3B, HepG2 and HepJ5 cells. GES1, GES6 and G5S1 scFv antibodies at concentrations of 100 μg/ml exhibited various but significant binding signal to all 3 hepatoma cells (left to central panels). Experiments performed using an irrelevant scFv antibody NC) showed little or no binding activity.

### Example 7 Immunofluorescence Staining Assay of the Anti-GPC Antibodies

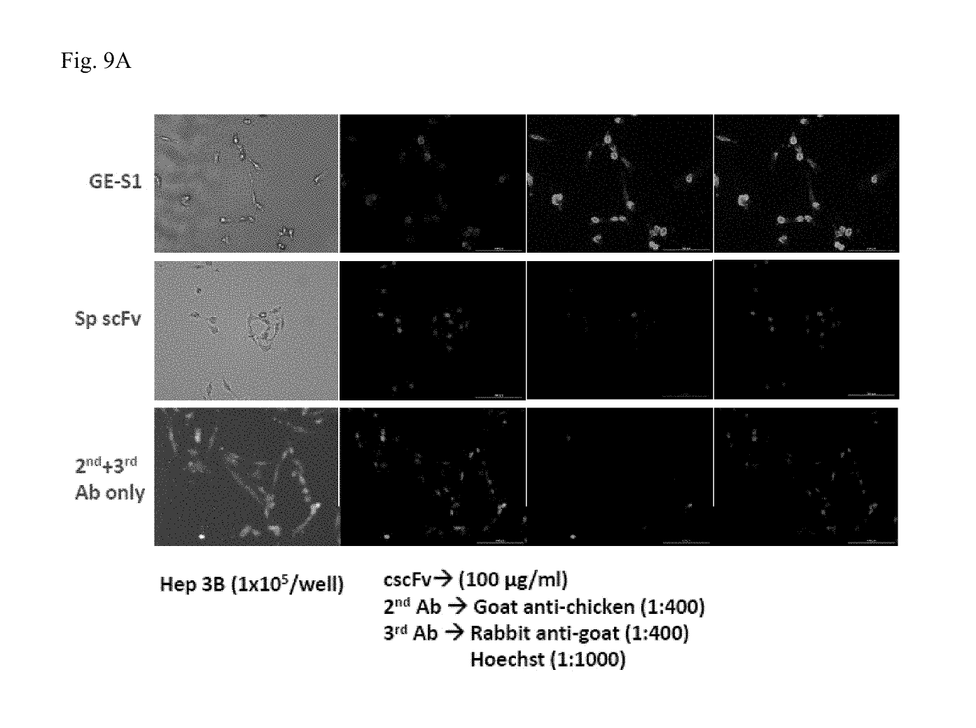

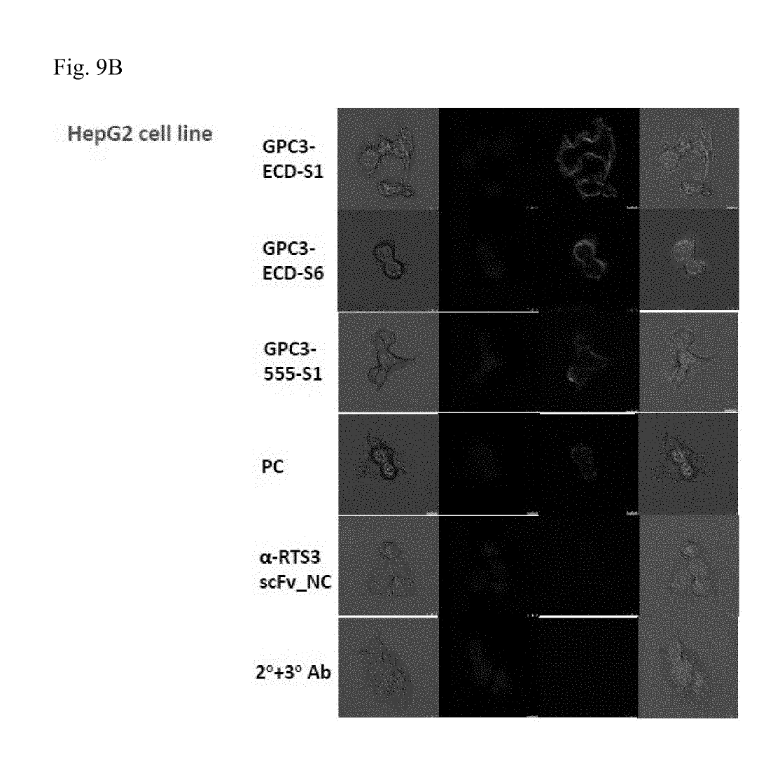

HepG2, Hep3B, cells (2×10⁵ cells/ml) were seeded on cover glass and fixed by incubating with equal volume of 8% freshly prepared ice-cold paraformaldehyde on ice for 15 min. After fixing, the cells were dehydrated in a sequential treatment of 70%, 95% and 99% methanol and rehydrated with 95% and 70% methanol. The slides were then overlaid with blocking buffer (1% BSA in 1×PBS) at room temperature (RT) for 1 hr. Following washing with 1×PBS, specific monoclonal mouse or scFv antibodies were incubated with cells at RT for one additional hr. Finally, their binding to GPC3 protein was detected by mouse anti-HA antibodies, followed by FITC-conjugated goat anti-mouse antibodies. Nuclei were counterstained with PI solution in parallel. The slides were examined using a Confocal Spectral Microscope Imaging System (TCS SP5, Leica). FIGS. 9 A and B shows the binding analysis of specific anti-GPC3 scFv antibodies using immunofluoresence staining. The partially purified scFv antibodies were examined for their binding activity to GPC3-positive HepG2 cells and Hep 3B cells. GES1, GES6 and G5S1 scFv antibodies exhibited various but significant binding signal to the surface of Hep 3B cells (FIG. 9A) and Hep G2 cells (FIG. 9B), which was comparable to that of polyclonal IgY antibodies from 4th-immunized chicken. Similar experiments performed with irrelevant scFv antibodies (Sp scFv in the FIG. 9A and α-RTS3 in the FIG. 9B) or with no addition of specific anti-GPC3 antibodies (2_{nd}+3_{rd} Ab only) showed no binding activity.

### Example 8 Immunoprecipitation Assay of the Anti-GPC Antibodies

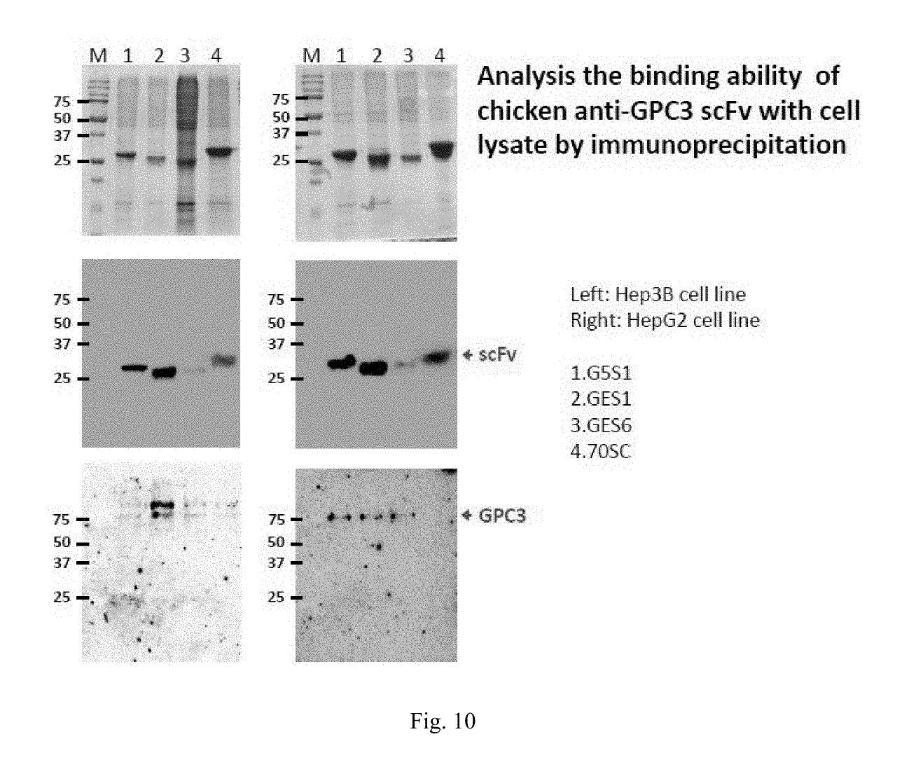

Antibody beads were prepared by covalently linking 100 ul of Ni-NTA Sepharose (Amersham Biosciences) and 1 mg of anti-GPC3 scFv antibodies with 20 mM dimethyl pimelimidate. Then, 500 ul of HepG2 and Hep3B cell lysates were be mixed with 25 ul of antibody beads and incubated for 2 hr at 4° C. After extensive washing with PBST, antibody beads will be resuspended in 50 ul of SDS-PAGE loading buffer, boiled for 5 min, and subsequently subjected to western blotting analysis. FIG. 10 shows the binding analysis of commercial anti-GPC3 scFv antibodies using immunoprecipitation analysis. The total cell lysates of Hep3B and HepG2 were incubated with a mixture of his-beads and G5S1, GES1, GES6 or 70SC control scFv antibodies individually. After washing, the his-beads complex was subjected to SDS-PAGE and western blot analysis. The results showed that a protein in Hep3B cells with the predicted molecular weight of GPC3 was precipitated by GES1. The results using the other 2 scFv antibodies, G5S1 and GES6, were not as obvious. By contrast, the protein in HepG2 cells was precipitated by GES1, G5S1 and GES6 scFv antibodies. However, the data were very preliminary and need further verification.

### Example 9 Soft Agar Assay of the Anti-GPC scFv Antibodies

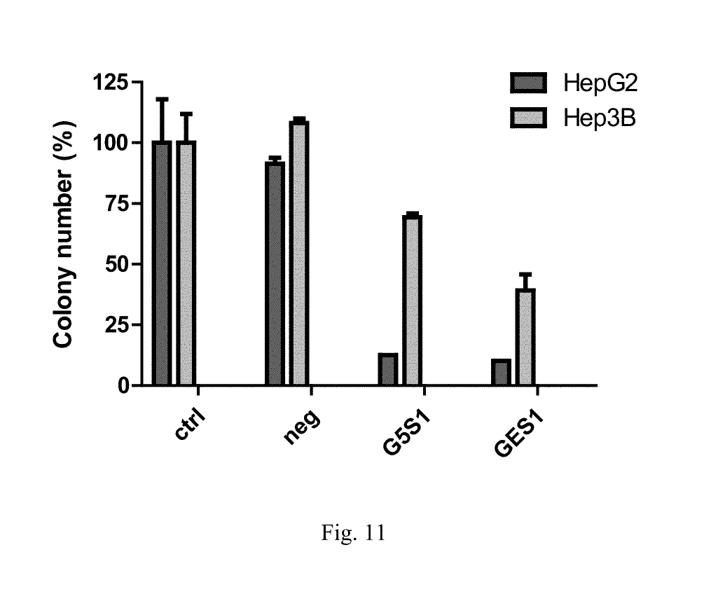

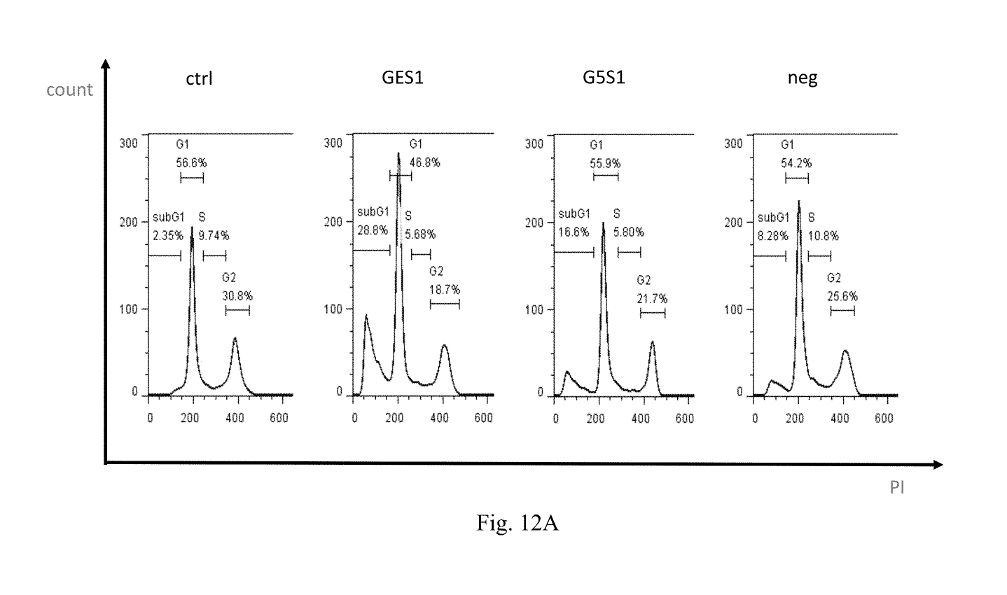

To determine the clonogenicity of HepG2, Hep3B, cells, 5×10⁵ cells were pre-incubated in 2 ml DMEM medium containing anti-GPC3 antibodies at concentrations of 0.5-5 uM for 48 hr. Thereafter, cells were harvested, counted and transferred into semi-solid medium supplemented with 0.8% DMEM-methylcellulose and 30% FBS. Finally, 1 ml of the semi-solid medium containing 5×10³ individual type of cell was plated onto 3.5 cm Petri-dishes. Triplicated Petri-dishes per treatment protocol will be cultivated for 5-7 days under standard conditions. Colony formation (clusters of >30 cells) will be visualized by staining with crystal violet and scored by an inverted microscope. FIG. 11 shows the inhibition of specific anti-GPC3 scFv antibodies on colony formation. The effect of GPC3 scFv on anchorage independent growth of HepG2 and Hep3B cells was explored. Hepatoma cells were treated with 0.5 μM of G5S1, GES1 or α-RTS6 (neg) scFv antibodies for 1 hr and suspended in medium containing 0.4% low melting agarose and plated onto solidified 0.9% agarose in medium at a density of 1×10⁵ cells per dish. After incubating for 4 weeks, the number of colonies was counted and recorded. Both G5S1 and GES1 scFv antibodies exhibited significant inhibitory effort on colony formation of HepG2 cells. Their inhibitory effect on Hep3B cells was not as obvious. The exact reason for the distinct inhibition on 2 cell lines is presently not known.

### Example 10 Cell Cycle Analysis

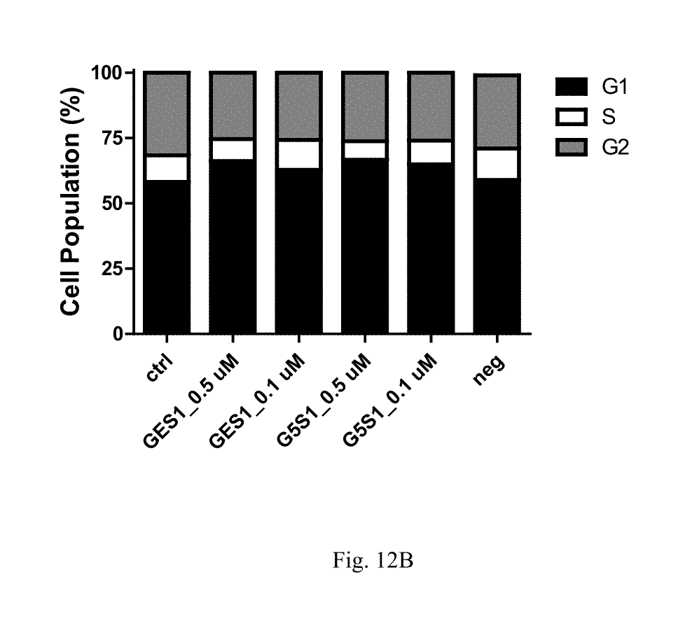

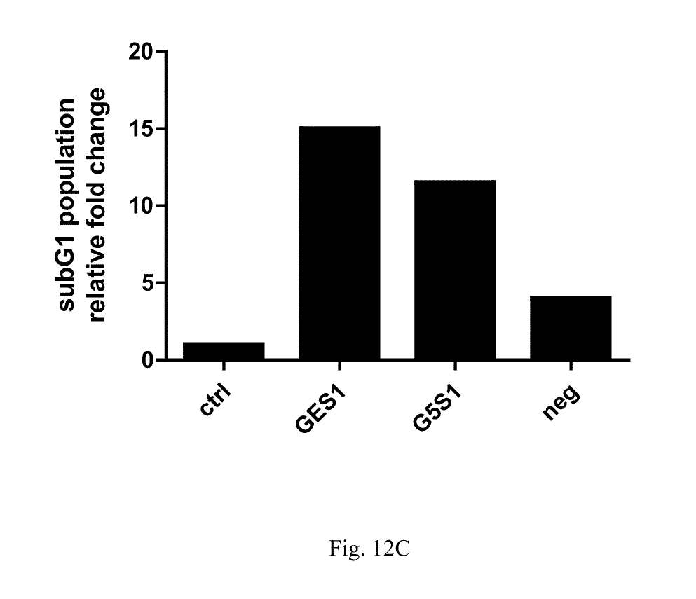

Cells were treated with 1 μM anti-GPC3 scFv antibodies for 2-5 days, collected and fixed with 70% (vol/vol) ethanol overnight. Fixed cells were stained with propidium iodide and analyzed by FACS. The cell-cycle distribution of different phases was analyzed with FlowJo v 9.0. FIGS. 12 A, B and C show the results of cell cycle analysis. After treated with G5S1, GES1 or α-RTS6 (neg) scFv antibodies for 48 hrs, the adherent HepG2 cells were detached, washed, and exposed to 70% ethanol on 4° C. overnight. After washing, cells were incubated with 5 mg/mL propidium iodide and 50 mg/mL RNaseA. FACS was carried out and data were statistically analyzed by FlowJo software. The cells were arrested in G1 phase when treated with 0.5 μM of G5S1 and GES1 scFv antibodies (FIGS. 12 A and B). In addition, the cell population in subG1 stage was significantly increased to 28.8% and 16.6% in GES1 and G5S1 treated HepG2 cells, respectively, which may result from the induction of cell apoptosis (FIG. 12 C).

### Example 11 Migration Assay

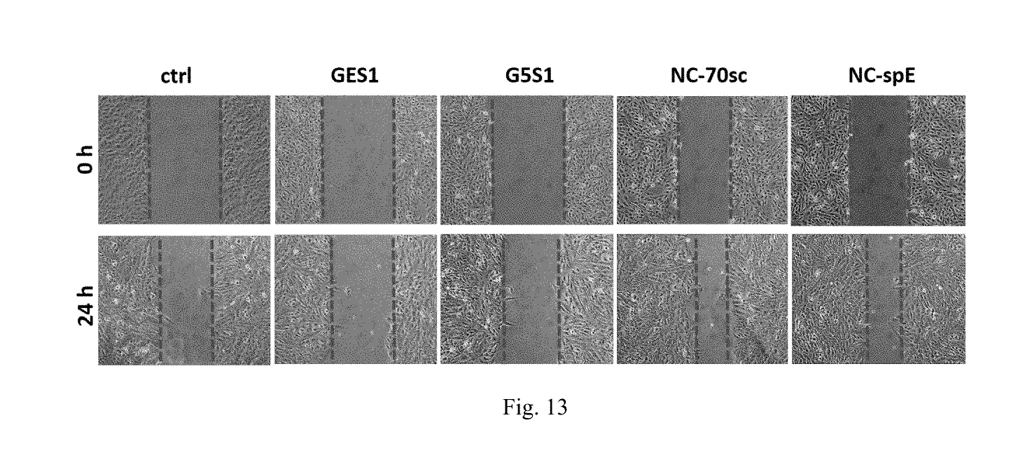

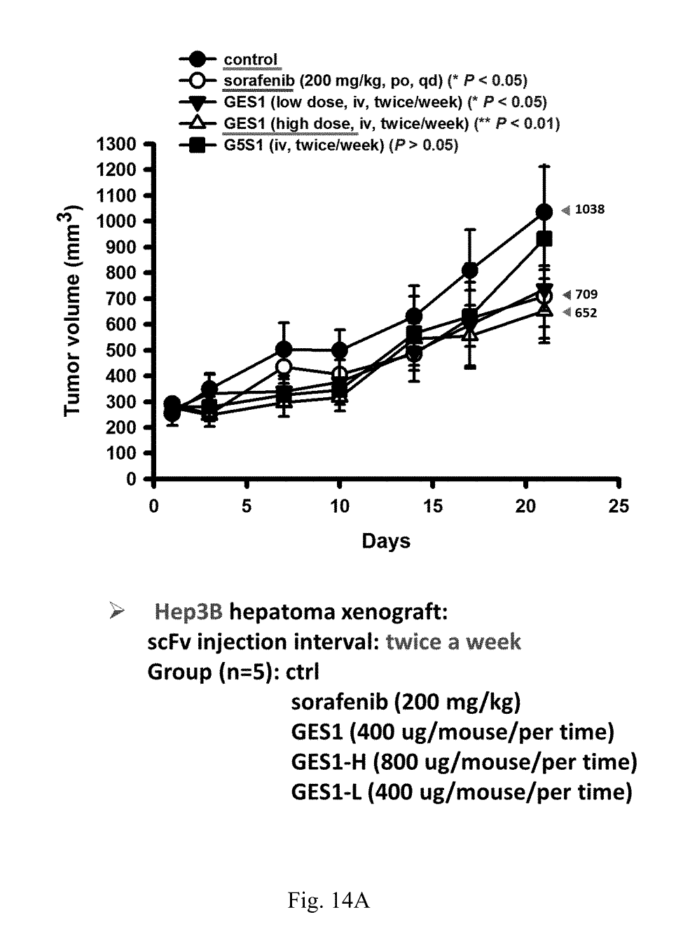

For cell migration assays, 1×10³ cells were incubated with anti-GPC3 antibodies at concentrations of 1 to 200 μg/ml for 48 hr before being transferred into a transwell migration system. The cells were plated onto a polycarbonate filter membrane with a pore size of 8 μm (upper layer) and incubated for different time interval at 37° C. in 5% CO₂ incubator. Serum free medium containing 0.5 mM EDTA was used for washing off the cells from the lower part of the membrane to lower wells. The cells in the lower wells were collected with centrifugation at 3000 g for 5 min and perform direct cell counting under microscope. The mean growth rate (MGR) of cells after migration through the polycarbonate filter was determined by the equation: MGR=log 2 Nt−log₂ N0/t, with N0 as initial cell number, Nt as final cell number and t as time period of cell incubation in days. FIG. 13 shows the inhibition of specific anti-GPC3 scFv antibodies on cell migration. To explore whether the GPC3 protein is essential for cell migration, Hep3B cells were cultured until confluence. The monolayers were wounded, cultured with medium containing 0.5 uM of G5 S1 or GES1 and two controls (NC-70sc or NC-spE) scFv antibodies and analyzed after 24, hr. A partial inhibitory effect on cell migration was observed when GES1 was used. The effect of G5S1 scFv was minimal and reached to the basal level obtained in medium without any scFv antibody. Intriguingly, an enhanced effect on cell migration was seen in medium containing negative control NC-70sc or NC-spE antibodies. These results need further investigation.

### Example 12 Tumor Xenograft Model and Immunohistochemical Analysis on Tumorous Tissues in Xenographic Mice

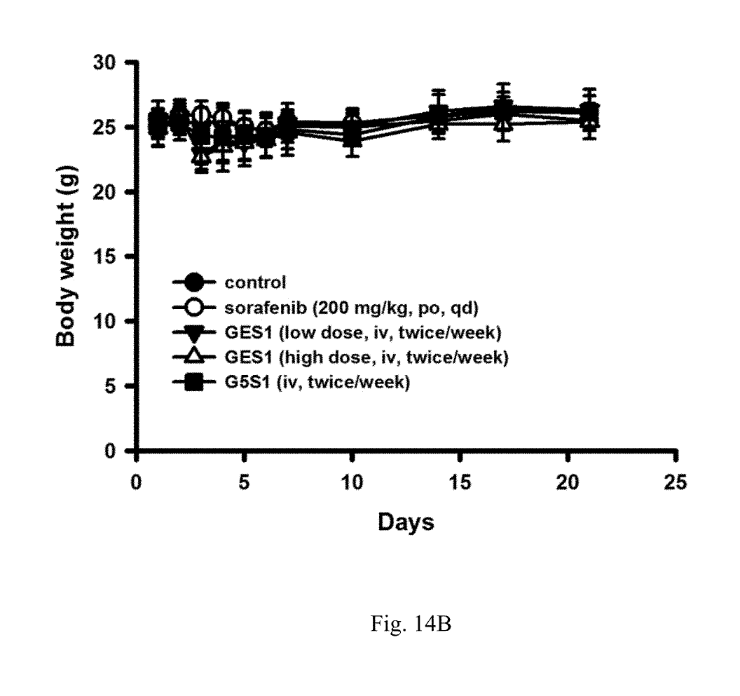

NOD-SCID mice were used. Briefly, 4-week-old female nude mice were injected subcutaneously with 4×10⁶ of HepG2 and Hep3B cancer cells at a single dorsal site. At day 14, tumor-bearing mice were randomized into experimental groups (5 per group) and treated with anti-GPC3 antibodies and positive control with sorafenib accordingly. 400 ug or 800 ug of antibodies were given through intravenous (i.v.) injection every 3 days for 3 weeks. Tumor size was measured every 2 days with calipers until animals are sacrificed. At the time of sacrifice, tumors were dissected and weighted. The antitumor effects and body weight change of G5S1 and GES1 against human Hep3B xanograft model are shown in FIG. 14.

### Example 12 Immunohistochemical Analysis on Tumorous Tissues in Xenographic Mice

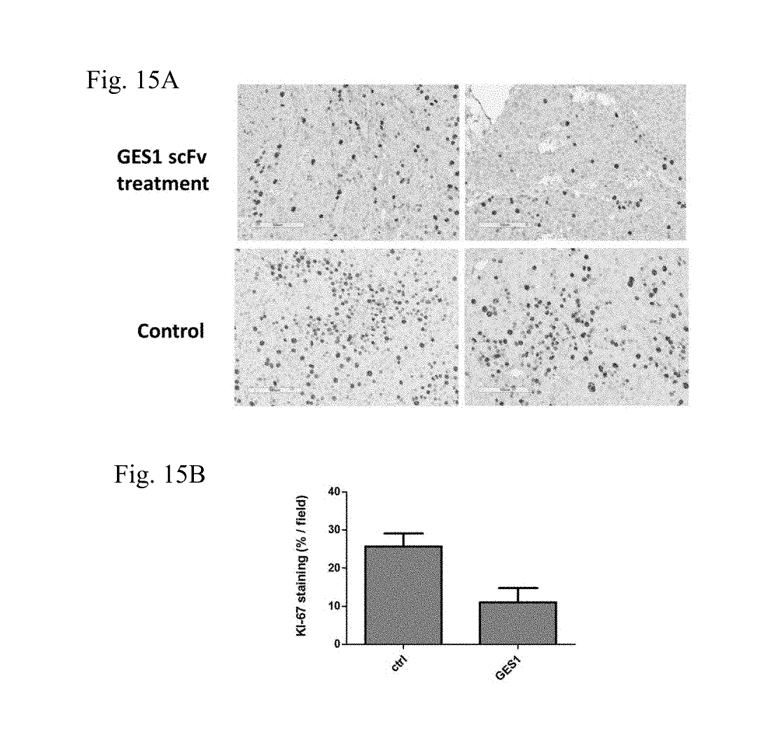

Tissue sections from xenograft tumor tissues in mice were de-waxed in xylene and rehydrated through graded alcohols. The antigen retrieval was carried out by heating the rehydrated tissues in 10 mM sodium citrate (pH 6.0) for 20 min. After washing with buffer containing 10 mM Tris-HCl (pH 7.4) and 150 mM sodium chloride, the sections were treated with 3% hydrogen peroxide for 5 min. Anti-Ki-67 antibodies (commercial polyclonal antibody) was be applied for 1 hr at room temperature. Optimal horseradish peroxidase-conjugated secondary antibodies and diaminobenzidine were added sequentially to detect the Ki-67 proliferation marker expression in the examined tissues. The slides were counterstained with GM hematoxylin solution. FIG. 15 shows immunohistochemical analysis on tumorous tissues in xenographic mice. After sacrificed, a portion of tumor samples was harvested and examined for the expression of Ki-67 protein, which is a marker commonly used to evaluate the cell proliferation. The results indicated that a reduced level of Ki-67 expression was detected in the tumor sections from GES1 (800 ug/mouse/per time) treated mice as compared to those from untreated groups.

### Example 13 Xanograft Animal Model

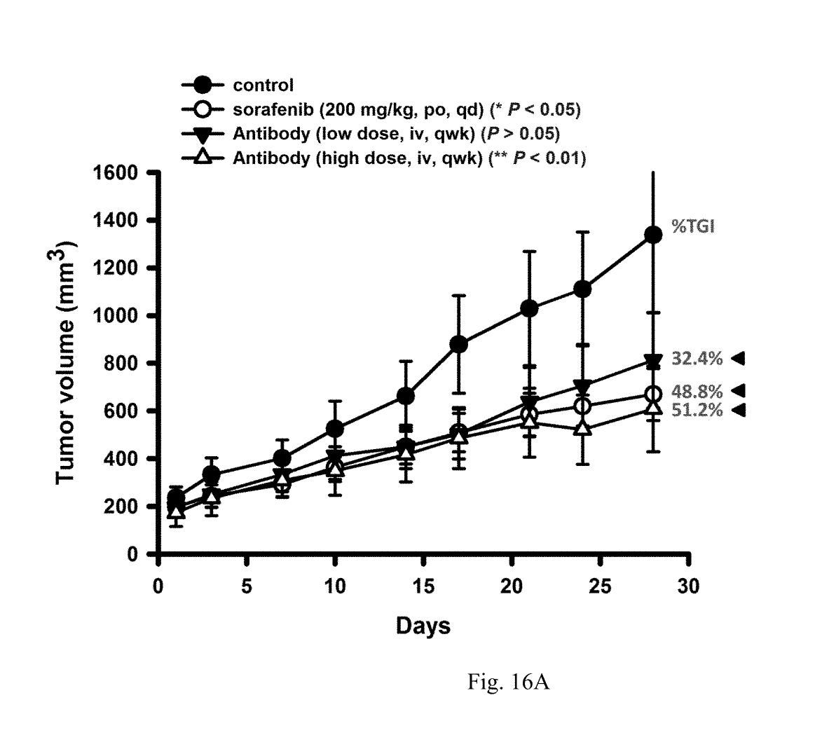

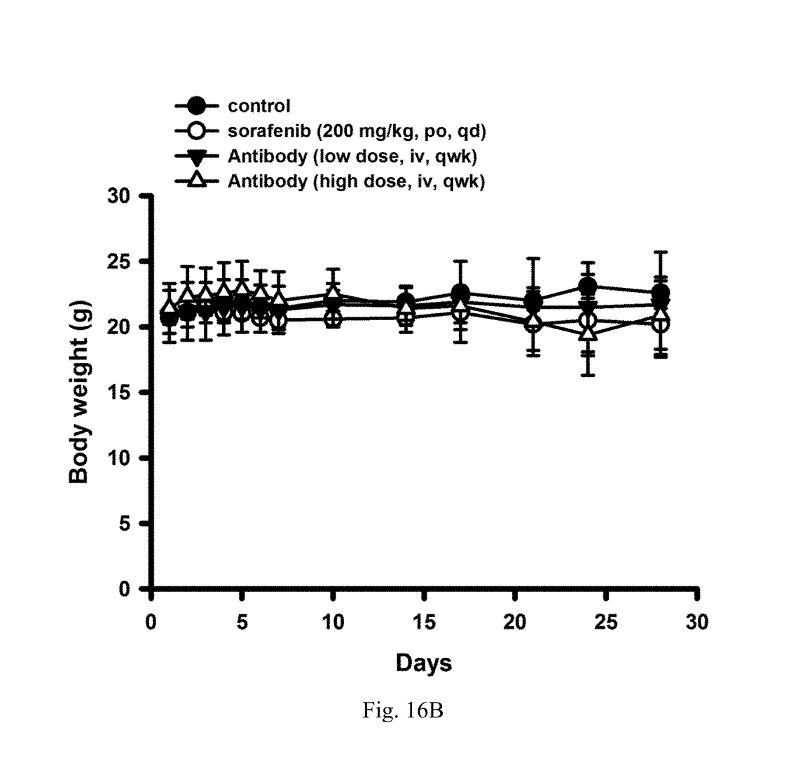

GES1 scFv antibody was humanized to obtain humanized IgG antibody (GES1 humanized scFv antibody) and a xenograft animal model was conducted. Hep3B hepatoma cells were cloned to NOD-SCID mice. After the tumors were generated, 1 mg/Kg and 5 mg/Kg of GES1 IgG were i.v. injected into the mice once a week. Another group of the mice were orally administered with 200 mg/Kg sorafenic. As shown in FIG. 16, 1 mg/Kg and 5 mg/Kg of GES1 IgG can inhibit tumor growth to 32.4% and 51.2%, respectively, whereas sorafenib only produces 48.8% inhibition. The results show that 1 mg/Kg of GES1 IgG exhibits an advantageous effect in inhibition of tumors and 5 mg/Kg of GES1 IgG exhibits higher tumor inhibition than sorafenib. There is no significant change in body weight of the mice.

### Example 14 Orthotropic Animal Model

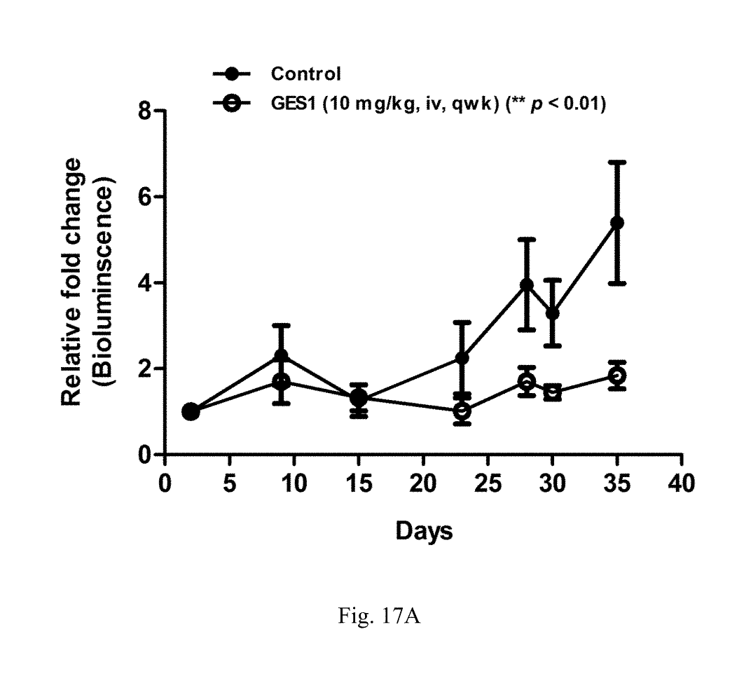

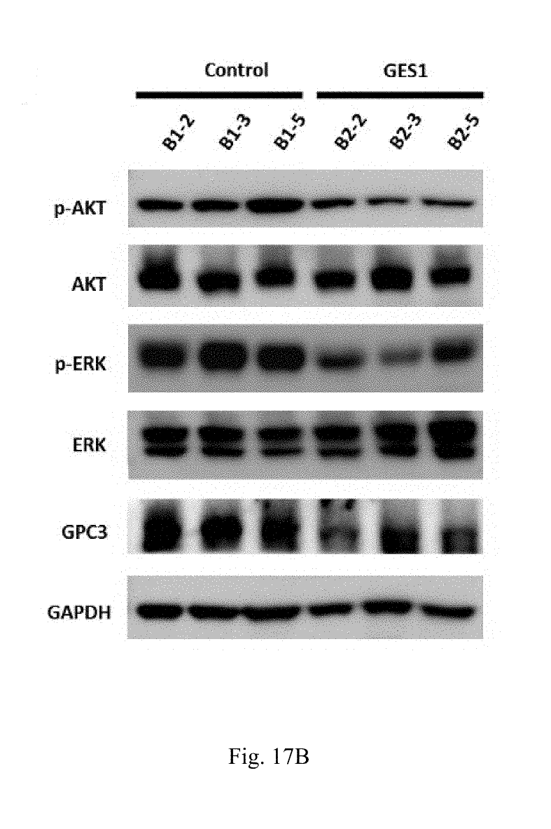

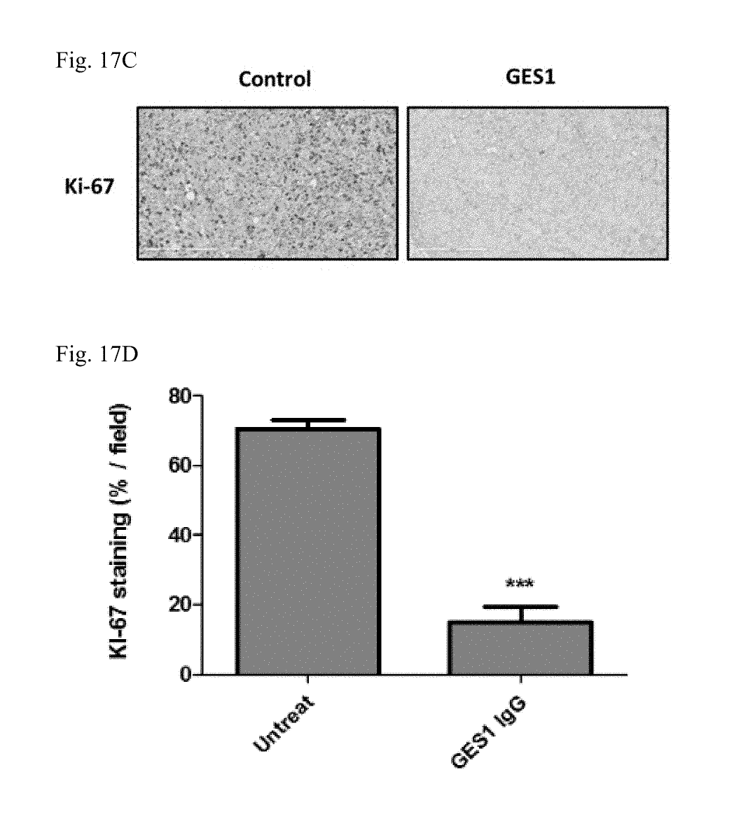

The orthotropic animal model with PanC1 tumor cells was conducted. The PanC1 cancer cells were inoculated into nude mice. After the tumors were generated, 10 mg/Kg of GES1 IgG were i.v. injected into two groups of the mice once a week. As shown in FIG. 17 A, 10 mg/Kg of GES1 IgG can significantly inhibit tumor growth (p<0.01). After isolation of tumor tissue proteins, the proteins were isolated and purified by Western blotting. As shown in FIG. 17B, the expression levels of p-AKT and p-Erk after antibody treatment decreased (B2-2, B2-3 and B2-5). The expression level of the target GPC3 of the antibody also decreased. In addition, as shown in FIGS. 17C and D, the expression level of Ki-67 protein in tumor tissues treated by GES1 IgG significantly decreased in comparison with those treated by the commercial anti-Ki-67 antibody.

Antibodies described in the examples for detecting Akt, Erk, p-Akt, p-Erk were purchased from Cell Signaling Technology, Inc. Antibody described in the examples for detecting GPC3 was purchased from Aviva Systems Biology, Corp. Antibody described in the examples for detecting Ki-67 was purchased from (Dako).

We claim:

1. An isolated anti-GPC3 antibody or an antigen-binding portion thereof, comprising a heavy chain complementarity determining region 1 (H-CDR1) comprising the amino acid residue of SEQ ID NO: 1, 2, 3 or 4, a heavy chain CDR2 (H-CDR2) comprising the amino acid residue of SEQ ID NO: 5, 6, 7, 8 or 9, and a heavy chain CDR3 (H-CDR3) comprising the amino acid residue of SEQ ID NO: 10, 11, 12, 13 or 14, and
a light chain CDR1 (L-CDR1) comprising the amino acid residue of SEQ ID NO: 15, 16 or 17; a light chain CDR2 (L-CDR2) comprising the amino acid residue of SEQ ID NO: 18, 19, 20, 21 or 22, and a light chain CDR3 (L-CDR3) comprising the amino acid residue SEQ ID NO: 23, 24, 25, 26 or 27.

2. (canceled)

3. (canceled)

4. The isolated anti-GPC3 antibody or an antigen-binding portion thereof of claim 1, comprising:
a heavy chain complementarity determining region 1 (H-CDR1) comprising the amino acid residue of SEQ ID NO: 1, a heavy chain CDR2 (H-CDR2) comprising the amino acid residue of SEQ ID NO: 5 and a heavy chain CDR3 (H-CDR3) comprising the amino acid residue of SEQ ID NO: 10; and a light chain CDR1 (L-CDR1) comprising the amino acid residue of SEQ ID NO: 15, a light chain CDR2 (L-CDR2) comprising the amino acid residue of SEQ ID NO: 18 and a light chain CDR3 (L-CDR3) comprising the amino acid residue SEQ ID NO: 23.

5. (canceled)

6. The isolated anti-GPC3 antibody or an antigen-binding portion thereof of claim 1, comprising a heavy chain complementarity determining region 1 (H-CDR1) comprising the amino acid residue of SEQ ID NO: 2, a heavy chain CDR2 (H-CDR2) comprising the amino acid residue of SEQ ID NO: 6 and a heavy chain CDR3 (H-CDR3) comprising the amino acid residue of SEQ ID NO: 11; and a light chain CDR1 (L-CDR1) comprising the amino acid residue of SEQ ID NO: 15, a light chain CDR2 (L-CDR2) comprising the amino acid residue of SEQ ID NO: 19 and a light chain CDR3 (L-CDR3) comprising the amino acid residue of SEQ ID NO: 24.

7. The isolated anti-GPC3 antibody or an antigen-binding portion thereof of claim 1, comprising a heavy chain complementarity determining region 1 (H-CDR1) comprising the amino acid residue of SEQ ID NO: 3, a heavy chain CDR2 (H-CDR2) comprising the amino acid residue of SEQ ID NO: 7 and a heavy chain CDR3 (H-CDR3) comprising the amino acid residue of SEQ ID NO: 12; and a light chain CDR1 (L-CDR1) comprising the amino acid residue of SEQ ID NO: 16, a light chain CDR2 (L-CDR2) comprising the amino acid residue of SEQ ID NO: 20 and a light chain CDR3 (L-CDR3) comprising the amino acid residue of SEQ ID NO: 25.

8. The isolated anti-GPC3 antibody or an antigen-binding portion thereof of claim 1, comprising a heavy chain complementarity determining region 1 (H-CDR1) comprising the amino acid residue of SEQ ID NO: 4, a heavy chain CDR2 (H-CDR2) comprising the amino acid residue of SEQ ID NO: 8 and a heavy chain CDR3 (H-CDR3) comprising the amino acid residue of SEQ ID NO: 13; and a light chain CDR1 (L-CDR1) comprising the amino acid residue of SEQ ID NO: 15, a light chain CDR2 (L-CDR2) comprising the amino acid residue of SEQ ID NO: 21 and a light chain CDR3 (L-CDR3) comprising the amino acid residue of SEQ ID NO: 26.

9. The isolated anti-GPC3 antibody or an antigen-binding portion thereof of claim 1, comprising a heavy chain complementarity determining region 1 (H-CDR1) comprising the amino acid residue of SEQ ID NO: 3, a heavy chain CDR2 (H-CDR2) comprising the amino acid residue of SEQ ID NO: 9 and a heavy chain CDR3 (H-CDR3) comprising the amino acid residue of SEQ ID NO: 14; and a light chain CDR1 (L-CDR1) comprising the amino acid residue of SEQ ID NO: 17, a light chain CDR2 (L-CDR2) comprising the amino acid residue of SEQ ID NO: 22 and a light chain CDR3 (L-CDR3) comprising the amino acid residue of SEQ ID NO: 27.

10. The isolated anti-GPC3 antibody or an antigen-binding portion thereof of claim 1, which is a monoclonal antibody, chimeric antibody, humanized antibody or human antibody.

11.-30. (canceled)

31. An isolated anti-GPC3 antibody or an antigen-binding portion thereof, comprising a heavy chain having an amino acid sequence as set forth in SEQ ID NO: 28 (G5S1 (555S1)) and a light chain having an amino acid sequence as set forth in SEQ ID NO: 36 (G5S1 (555S1)), a heavy chain having an amino acid sequence as set forth in SEQ ID NO: 29 (G5S8 (555S8)) and a light chain having an amino acid sequence as set forth in SEQ ID NO: 37 (G5S8 (555S8)), a heavy chain having an amino acid sequence as set forth in SEQ ID NO: 30 (GES1 (GPC3 S1)) and a light chain having an amino acid sequence as set forth in SEQ ID NO: 38 (G5S8 (555S8)), a heavy chain having an amino acid sequence as set forth in SEQ ID NO: 31 (GES2 (GPC3 S2)) and a light chain having an amino acid sequence as set forth in SEQ ID NO: 39 (G5S8 (555S8)), a heavy chain having an amino acid sequence as set forth in SEQ ID NO: 32 (GES6 (GPC3 S6)) and a light chain having an amino acid sequence as set forth in SEQ ID NO: 40 (G5S8 (555S8)), or a heavy chain having an amino acid sequence as set forth in SEQ ID NO: 33 (GES8 (GPC3 S8)) and a light chain having an amino acid sequence as set forth in SEQ ID NO: 41 (G5S8 (555S8)).

32. The isolated antibody of claim 1, which is a humanized scFv antibody.

33. The isolated antibody of claim 32, wherein the humanized scFv antibody comprises a heavy chain having an amino acid sequence as set forth in SEQ ID NO: 34 and a light chain having an amino acid sequence as set forth in SEQ ID NO: 42 (G5S1 humanized scFv antibody).

34. The isolated antibody of claim 32, wherein the humanized scFv antibody comprises a heavy chain having an amino acid sequence as set forth in SEQ ID NO: 35 and a light chain having an amino acid sequence as set forth in SEQ ID NO: 43 (GES1 humanized scFv antibody).

35. A pharmaceutical composition comprising an antibody of claim 1 and a pharmaceutically acceptable carrier.

36. The pharmaceutical composition of claim 35, which comprises an additional anti-tumor drug.

37. A method for treating a cancer in a subject comprising administering an effective amount of the anti-GPC3 antibody of claim 1 to the subject in need thereof.

38. The method of claim 37, wherein the cancer is cancer of the liver, skin,
head and neck, lung, breast, prostate, ovaries, endometrium, cervix, colon, rectum, bladder, brain, stomach, pancreas or lymphatic system.

39. The method of claim 37, wherein the cancer is liver cancer such as hepatocellular carcinoma (HCC), hepatoblastoma and sarcomatoid HCC.

40. (canceled)

41. A method for diagnosing a cancer in a subject, comprising detecting the binding of the anti-GPC3 antibody of claim 1 to GPC3 in a biological sample, wherein the binding indicates the likelihood of the subject developing a cancer.

42. The method of claim 41, wherein the cancer is ovarian cancer, breast cancer, liver cancer, lung cancer, non-small cell lung cancer, small cell lung cancer (including small cell carcinoma (oat cell cancer), mixed small cell/large cell carcinoma, and combined small cell carcinoma), colon cancer, prostate cancer, pancreatic cancer, brain cancer, kidney cancer, stomach cancer, melanoma, bone cancer, gastric cancer, breast cancer, glioma, gliobastoma, hepatocellular carcinoma, papillary renal carcinoma, head and neck squamous cell carcinoma, leukemia, lymphoma or myeloma.

43. A method for diagnosing a cirrhotic liver or liver cancer in a subject, comprising detecting the binding of the anti-GPC3 antibody of claim 1 to GPC3 in a biological sample, wherein the binding indicates the likelihood of the subject developing a cirrhotic liver and liver cancer.

44. (canceled)

45. A diagnostic agent or kit for diagnosing a cancer, cirrhotic liver or liver cancer comprising anti-GPC3 antibody of claim 1.


SEQUENCE LISTING (38 sequences)

SEQ ID NO: 1 - protein, 8 aa, Artificial Sequence
  Synthetic polypeptide
GFTFSSYA

SEQ ID NO: 2 - protein, 8 aa, Artificial Sequence
  Synthetic polypeptide
GFTFSSVN

SEQ ID NO: 3 - protein, 8 aa, Artificial Sequence
  Synthetic polypeptide
GFTFSSFN

SEQ ID NO: 4 - protein, 8 aa, Artificial Sequence
  Synthetic polypeptide
GFTFNNYC

SEQ ID NO: 5 - protein, 8 aa, Artificial Sequence
  Synthetic polypeptide
VSKDGTTT

SEQ ID NO: 6 - protein, 7 aa, Artificial Sequence
  Synthetic polypeptide
ISNTNTT

SEQ ID NO: 7 - protein, 8 aa, Artificial Sequence
  Synthetic polypeptide
ISSTGSRT

SEQ ID NO: 8 - protein, 8 aa, Artificial Sequence
  Synthetic polypeptide
ISKDGSTP

SEQ ID NO: 9 - protein, 8 aa, Artificial Sequence
  Synthetic polypeptide
ISGTGSST

SEQ ID NO: 10 - protein, 15 aa, Artificial Sequence
  Synthetic polypeptide
AKSNTNSRAAGLIDA

SEQ ID NO: 11 - protein, 16 aa, Artificial Sequence
  Synthetic polypeptide
ARGSGVSGTYAGQIDA

SEQ ID NO: 12 - protein, 13 aa, Artificial Sequence
  Synthetic polypeptide
AKSASRGAGRIDA

SEQ ID NO: 13 - protein, 17 aa, Artificial Sequence
  Synthetic polypeptide
ARGGGSNYCGSTGRINA

SEQ ID NO: 14 - protein, 16 aa, Artificial Sequence
  Synthetic polypeptide
AKGVDSDSWTAAGIDA

SEQ ID NO: 15 - protein, 5 aa, Artificial Sequence
  Synthetic polypeptide
SGSYG

SEQ ID NO: 16 - protein, 5 aa, Artificial Sequence
  Synthetic polypeptide
GGSYG

SEQ ID NO: 17 - protein, 6 aa, Artificial Sequence
  Synthetic polypeptide
TGRWYG

SEQ ID NO: 18 - protein, 10 aa, Artificial Sequence
  Synthetic polypeptide
GSRDSSYVGI

SEQ ID NO: 19 - protein, 14 aa, Artificial Sequence
  Synthetic polypeptide
GNYDGNTDSGYVGV

SEQ ID NO: 20 - protein, 10 aa, Artificial Sequence
  Synthetic polypeptide
GSRDSSYFGI

SEQ ID NO: 21 - protein, 11 aa, Artificial Sequence
  Synthetic polypeptide
GNAGSSTYAGI

SEQ ID NO: 22 - protein, 11 aa, Artificial Sequence
  Synthetic polypeptide
GSRESSRNPGI

SEQ ID NO: 23 - protein, 122 aa, Artificial Sequence
  Synthetic polypeptide
AVTLDESGGGLQTPGGGLSLVCKASGFTFSSYAMQWVRQAPGKGLEWVAGVSKDGTTTKYGSAVQGRATISRDNGQSTVRLQLNNLRAEDTATYYCAKSNTNSRAAGLIDAWGHGTEVIVSS

SEQ ID NO: 24 - protein, 122 aa, Artificial Sequence
  Synthetic polypeptide
TVTLDESGGGLQTPGGGLSLVCKASGFTFSSVNMFWVRQAPGKGLEFVAGISNTNTTGYAAAVKGRATITRDNGQSTVRLQLNNLRAEDTGIYYCARGSGVSGTYAGQIDAWGHGTEVIVSS

SEQ ID NO: 25 - protein, 120 aa, Artificial Sequence
  Synthetic polypeptide
AVTLDESGGGLQTPGGALSLVCKGSGFTFSSFNMFWVRQAPGKGLEYVAQISSTGSRTYYAPAVKGRATISRDNGQNTVRLQLNNLRAEDTATYYCAKSASRGAGRIDAWGHGTEVIVSS

SEQ ID NO: 26 - protein, 120 aa, Artificial Sequence
  Synthetic polypeptide
AVTLDESGGGLQTPGGALSLVCKGSGFTFSSFNMFWVRQAPGKGLEYVAQISSTGSRTYYAPAVKGRATISRDNGQSTVRLQLNNLRAEDTATYYCAKSASRGAGRIDAWGHGTEVIVSS

SEQ ID NO: 27 - protein, 124 aa, Artificial Sequence
  Synthetic polypeptide
AVTLDESGGGLQTPGGTLSLVCKASGFTFNNYCMNWVRQAPGKGLEWVAAISKDGSTPYYAPAVKGRATISRDDGQSTVRLQLNNLRADDTGTYFCARGGGSNYCGSTGRINAWGHGTEVIVSS

SEQ ID NO: 28 - protein, 123 aa, Artificial Sequence
  Synthetic polypeptide
TVTLDESGGGLQLPGGALSLVCKGSGFTFSSFNMFWVRQAPGKGLEYVAEISGTGSSTYYAPAVKGRATISRDNGQSTVRLQLNNLRAEDTGTYYCAKGVDSDSWTAAGIDAWGHGTEVIVSS

SEQ ID NO: 29 - protein, 122 aa, Artificial Sequence
  Synthetic polypeptide
QVQLVESGGGLVQPGGSLRLSCAASGFTFSSYAINWVRQAPGKGLEWVSGVSKDGTTTLYADSVKGRFTISRDNSKNTLYLQMNSLRAEDTAVYYCAKSNTNSRAAGLIDAWGQGTLVTVSS

SEQ ID NO: 30 - protein, 120 aa, Artificial Sequence
  Synthetic polypeptide
EVQLLESGGGLVQPGGSLRLSCAASGFTFSSFNMFWVRQAPGKGLEWVSWISSTGSRTKYADSVKGRFTISRDNSKNTLYLQMNSLRAEDTATYYCAKSASRGAGRIDAWGRGTLVTVSS

SEQ ID NO: 31 - protein, 103 aa, Artificial Sequence
  Synthetic polypeptide
ALTQPSSVSANLGGTVKITCSGGSGSYGWYQQKSPGSAPVTLIYANTNRPSDIPSRFSGSKSGSTSTLTITGVQAEDVAVYYCGSRDSSYVGIFGAGTTLTVL

SEQ ID NO: 32 - protein, 107 aa, Artificial Sequence
  Synthetic polypeptide
ALTQPSLVSANLGGTAEITCSGGSGSYGWYQQKSPGSAPVTVIYQNDKRPSDIPSRFSGSKSGSTGTLTITGVQVEDEAVYYCGNYDGNTDSGYVGVFGAGTTLTVL

SEQ ID NO: 33 - protein, 103 aa, Artificial Sequence
  Synthetic polypeptide
ALTQPSSVSANPGETVKITCSGGGGSYGWYQQKSPGSAPVTVIHNNNNRPSDIPSRFSGSKSGSTGTLTITGVQAEDEAVYYCGSRDSSYFGIFGAGTTLTVL

SEQ ID NO: 34 - protein, 103 aa, Artificial Sequence; identical to SEQ ID NO: 33
  Synthetic polypeptide

SEQ ID NO: 35 - protein, 104 aa, Artificial Sequence
  Synthetic polypeptide
ALTQPSSVSANLGGTVEITCSGGSGSYGWHQQKSPGSAPVTVIYRNNQRPSDIPSRFSGSKSGSTHTLTITGVRAEDEAVYFCGNAGSSTYAGIFGAGTTLTVL

SEQ ID NO: 36 - protein, 105 aa, Artificial Sequence
  Synthetic polypeptide
ALTQPSSVSANLGGTVKISCSGGTGRWYGWYQQKSPGSLPVTVIYSNDKRPSDIPSRFSGSTSGSTSTLTITGVQAEDEAVYFCGSRESSRNPGIFGAGTTLTVL

SEQ ID NO: 37 - protein, 106 aa, Artificial Sequence
  Synthetic polypeptide
ELALIQPASVSVSPGQTASITCSGDSGSYGASWYQQKPGQSPVLVIYANTERPSGIPERFSGSNSGNTATLTISGTQAMDEADYYCGSRDSSYVGIFGGGTTLTVL

SEQ ID NO: 38 - protein, 102 aa, Artificial Sequence
  Synthetic polypeptide
ALTQPPSASGSPGQSITISCTGTGGSYGWYQQHAGKAPKLVIYNNNKRPSGVPDRFSGSKSGNTASLTVSGLQTDDEAVYYCGSRDSSYFGIFGGGTKLTVL